SEQUENCE LISTING (36 sequences)

SEQ ID NO: 1 - protein, 375 aa, Homo sapiens
MSTAGKVIKCKAAVLWELKKPFSIEEVEVAPPKAHEVRIKMVAVGICGTDDHVVSGTMVTPLPVILGHEAAGIVESVGEGVTTVKPGDKVIPLAIPQCGKCRICKNPESNYCLKNDVSNPQGTLQDGTSRFTCRRKPIHHFLGISTFSQYTVVDENAVAKIDAASPLEKVCLIGCGFSTGYGSAVNVAKVTPGSTCAVFGLGGVGLSAIMGCKAAGAARIIAVDINKDKFAKAKELGATECINPQDYKKPIQEVLKEMTDGGVDFSFEVIGRLDTMMASLLCCHEACGTSVIVGVPPDSQNLSMNPMLLLTGRTWKGAILGGFKSKECVPKLVADFMAKKFSLDALITHVLPFEKINEGFDLLHSGKSIRTILMF

SEQ ID NO: 2 - protein, 348 aa, Saccharomyces cerevisiae
MSIPETQKGVIFYESHGKLEYKDIPVPKPKANELLINVKYSGVCHTDLHAWHGDWPLPVKLPLVGGHEGAGVVVGMGENVKGWKIGDYAGIKWLNGSCMACEYCELGNESNCPHADLSGYTHDGSFQQYATADAVQAAHIPQGTDLAQVAPILCAGITVYKALKSANLMAGHWVAISGAAGGLGSLAVQYAKAMGYRVLGIDGGEGKEELFRSIGGEVFIDFTKEKDIVGAVLKATDGGAHGVINVSVSEAAIEASTRYVRANGTTVLVGMPAGAKCCSDVFNQVVKSISIVGSYVGNRADTREALDFFARGLVKSPIKVVGLSTLPEIYEKMEKGQIVGRYVVDTSK

SEQ ID NO: 3 - protein, 891 aa, Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 4 - protein, 374 aa, Equus caballus
MSAEVIKCKAAVAWEAGKPVSIEEVEVAPPKAHEVRIKIIATAVCHTDAYTLSGADPEGSFPVILGHEGAGIVESVGEGVTKLKAGDTVIPLYIPQCGECKFCLNPQTNLCQKIRTTQGKGLMPDGTSRFTCKGKTILHYMGTSTFSEYTVVADISVAKIDPLAPLDKVCLLGCGVSTGYGAAVNTAKVEPGSTCAIFGLGGVGLAVIMGCKVAGASRIIGVDINKDKFAKAKEFGASECINPQDFSKPIQEVLIEMTDGGVDYSFECIGNVKVMRAALEACHKGWGVSVVVGVAASGEEIATRPFQLVTGRTWKGTAFGGWKSVESIPKLVSEYMSKKIKVDEFVTHSLSFDQINEAFELMHAGKSIRTVVKL

SEQ ID NO: 5 - protein, 395 aa, Salmonella enterica
MQAELQTALFQAFDTLNLQRVKTFSVPPVTLCGLGALGACGQEAQARGVSHLFVMVDSFLHQAGMTAPLARSLAMKGVAMTVWPCPPGEPCITDVCAAVAQLREAACDGVVAFGGGSVLDAAKAVALLVTNPDQTLSAMTEHSTLRPRLPLIAVPTTAGTGSETTNVTVIIDAVSGRKQVLAHASLMPDVAILDAAVTEGVPPNVTAMTGIDALTHAIEAYSALNATPFTDSLAIGAIAMIGKSLPKAVGYGHDLEARENMLLASCMAGMAFSSAGLGLCHAMAHQPGAALHIPHGQANAMLLPTVMGFNRMVCRERFSQIGRALTNKKSDDRDAIAAVCELIAEVGQSKRLADAGAKPEHYSAWAQAALEDICLRSNPRTATQAQIIDLYAAAG

SEQ ID NO: 6 - protein, 623 aa, Pseudomonas aeruginosa
MTTRTSPAPAGLLRPSLHCLAFAVALGSAGAALAKDVTWEDIANDDKTTGDVLQYGMGTHAQRWSPLKQVNADNVFKLTPAWSYSFGDEKQRGQESQAIVSDGVIYVTASYSRLFALDAKTGKRLWTYNHRLPDDIRPCCDVVNRGAAIYGDKVFFGTLDASVVALNKNTGKVVWKKKFADHGAGYTMTGAPTIVKDGKTGKVLLIHGSSGDEFGVVGRLFARDPDTGEEIWMRPFVEGHMGRLNGKDSTVTGDVKAPSWPDDRNSPTGKVESWSHGGGAPWQSASFDAETNTIIVGAGNPGPWNTWARTAKGGNPHDYDSLYTSGQVGVDPSSGEVKWFYQHTPNDAWDFSGNNELVLFDYKAKDGKIVKATAHADRNGFFYVVDRSNGKLQNAFPFVDNITWASHIDLKTGRPVEREGQRPPLPEPGQKHGKAVEVSPPFLGGKNWNPMAYSQDTGLFYVPANHWKEDYWTEEVSYTKGSAYLGMGFRIKRMYDDHVGSLRAMDPVSGKVVWEHKEHLPLWAGVLATAGNLVFTGTGDGYFKAFDAKSGKELWKFQTGSGIVSPPITWEQDGEQYLGVTVGYGGAVPLWGGDMADLTRPVAQGGSFWVFKLPSWDNRTASR

SEQ ID NO: 7 - protein, 342 aa, Candida antarctica
MKLLSLTGVAGVLATCVAATPLVKRLPSGSDPAFSQPKSVLDAGLTCQGASPSSVSKPILLVPGTGTTGPQSFDSNWIPLSTQLGYTPCWISPPPFMLNDTQVNTEYMVNAITALYAGSGNNKLPVLTWSQGGLVAQWGLTFFPSIRSKVDRLMAFAPDYKGTVLAGPLDALAVSAPSVWQQTTGSALTTALRNAGGLTQIVPTTNLYSATDEIVQPQVSNSPLDSSYLFNGKNVQAQAVCGPLFVIDHAGSLTSQFSYVVGRSALRSTTGQARSADYGITDCNPLPANDLTPEQKVAAAALLAPAAAAIVAGPKQNCEPDLMPYARPFAVGKRTCSGIVTP

SEQ ID NO: 8 - protein, 356 aa, Homo sapiens
MGLLRIMMPPKLQLLAVVAFAVAMLFLENQIQKLEESRSKLERAIARHEVREIEQRHTMDGPRQDATLDEEEDMVIIYNRVPKTASTSFTNIAYDLCAKNKYHVLHINTTKNNPVMSLQDQVRFVKNITSWKEMKPGFYHGHVSYLDFAKFGVKKKPIYINVIRDPIERLVSYYYFLRFGDDYRPGLRRRKQGDKKTFDECVAEGGSDCAPEKLWLQIPFFCGHSSECWNVGSRWAMDQAKYNLINEYFLVGVTEELEDFIMLLEAALPRFFRGATELYRTGKKSHLRKTTEKKLPTKQTIAKLQQSDIWKMENEFYEFALEQFQFIRAHAVREKDGDLYILAQNFFYEKIYPKSN

SEQ ID NO: 9 - protein, 356 aa, Sus scrofa
MGLLRIMMPPKLQLLAVVAFAVAMLFLENQIQKLEESRSKLERAIARHEVREIEQRHTMDGPRQEAALDEEEDLVIIYNRVPKTASTSFTNIAYDLCAKNKYHVLHINTTKNNPVMSLQDQVRFVKNITSWKEMKPGFYHGHVSYLDFAKFGVKKKPIYINVVRDPIERLVSYYYFLRFGDDYRPGLRRRKQGDKKTFDECVAEGGSDCAPEKLWLQIPFFCGHSSECWNVGSRWAMDQAKYNLINEYFLVGVTEELEDFVMLLEAALPRFFRGATELYRTGKKSHLRKTTEKKLPTKQTIAKLQQSHIWKMENEFYEFALEQFQFIRAHAVREKDGDLFILAQNFFYEKIYPKSN

SEQ ID NO: 10 - protein, 356 aa, Gallus gallus
MGLLRIMLPPKLQLLAVLVFGVAVLFLENQIQKLEESRGKLERAIARHEVREIEQRHTADGPRQEVALDEEDDVVIIYNRVPKTASTSFTNIAYDLCAKNRYHVLHINTTKNNPVMSLQDQVRFVKNVTSWKEMKPGFYHGHVSYLDFAKFGVKKKPIYINVIRDPIERLVSYYYFLRFGDDYRPGLRRRKQGDKKTFDECVAAGGSDCAPEKLWLQIPFFCGHSSECWNVGSRWALEQAKYNLINEYFLVGVTEELEDFIMLLEAALPRFFRGATELYRTGKKSHLRKTTEKKLPTKETIAKLQQSEIWKMENEFYEFALEQFQFVRAHAVREKDGELYILAQNFFYEKIYPKSN

SEQ ID NO: 11 - protein, 312 aa, Homo sapiens
MKYYFPVRELERSLRFDMKGDDVIVFLHIQKTGGTTFGRHLVQNVRLEVPCDCRPGQKKCTCYRPNRRETWLFSRFSTGWSCGLHADWTELTNCVPGVLDRRDSAALRTPRKFYYITLLRDPVSRYLSEWRHVQRGATWKTSLHMCDGRTPTPEELPPCYEGTDWSGCTLQEFMDCPYNLANNRQVRMLADLSLVGCYNLSFIPEGKRAQLLLESAKKNLRGMAFFGLTEFQRKTQYLFERTFNLKFIRPFMQYNSTRAGGVEVDEDTIRRIEELNDLDMQLYDYAKDLFQQRYQYKRQLERREQRLRSREE

SEQ ID NO: 12 - protein, 312 aa, Sus scrofa
MKYYFPVRELERSLHFDMKGDDVIVFLHIQKTGGTTFGRHLVQNVRLEVPCDCRPGQKKCTCYRPNRRETWLFSRFSTGWSCGLHADWTELTNCVPGVLDRRDPAALRTPRKFYYITLLRDPVSRYLSEWRHVQRGATWKTSLHMCDGRTPTPEELPPCYEGTDWSGCTLQEFMDCPYNLANNRQVRMLADLSLVGCYNLSFIPEGKRSQLLLESAKKNLRGMAFFGLTEFQRKTQYLFERTFNLKFIRPFMQYNSTRAGGVEVGEDTIRRIEELNDLDMQLYDYARDLFQQRYQYKRQLERRQQRLRSREE

SEQ ID NO: 13 - protein, 311 aa, Gallus gallus
KYYFPVRELERELAFDMKGEDVIVFLHIQKTGGTTFGRHLVQNVRLEVPCDCRPGQKKCTCYRPNRRETWLFSRFSTGWSCGLHADWTELTNCVPGVLGRRESAPNRTPRKFYYITLLRDPVSRYLSEWRHVQRGATWKTSLHMCDGRTPTPEELPSCYEGTDWSGCTLQEFMDCPYNLANNRQVRMLADLSLVGCYNMSFIPENKRAQILLESAKKNLKDMAFFGLTEFQRKTQYLFERTFNLKFIRPFMQYNSTRAGGVEVDNDTIRRIEELNDLDMQLYDYAKDLFQQRYQYKRQLERMEQRIKNREE

SEQ ID NO: 14 - protein, 307 aa, Homo sapiens
MAALLLGAVLLVAQPQLVPSRPAELGQQELLRKAGTLQDDVRDGVAPNGSAQQLPQTIIIGVRKGGTRALLEMLSLHPDVAAAENEVHFFDWEEHYSHGLGWYLSQMPFSWPHQLTVEKTPAYFTSPKVPERVYSMNPSIRLLLILRDPSERVLSDYTQVFYNHMQKHKPYPSIEEFLVRDGRLNVDYKALNRSLYHVHMQNWLRFFPLRHIHIVDGDRLIRDPFPEIQKVERFLKLSPQINASNFYFNKTKGFYCLRDSGRDRCLHESKGRAHPQVDPKLLNKLHEYFHEPNKKFFELVGRTFDWH

SEQ ID NO: 15 - protein, 311 aa, Sus scrofa
MAALLLGAVMLVLQLQLVPCRPAMPGAGPSQQELVRKAATLQDEVRDSAAPNGSVQQLPQTIIIGVRKGGTRALLEMLSLHPDVAAAENEVHFFDWEEHYSQGLDWYLSQMPFSYPHQLTVEKTPAYFTSPKVPERVHRMNPSIRLLLILRDPSERVLSDYTQVFYNHVQKHKPYPSIEEFLVRDGRLNVDYKALNRSLYHVHMQNWLRFFPLRRIHIVDGDRLIRDPFPEIQKVERFLMLSPQINASNFYFNKTKGFYCLRDGGRDRCLHESKGRAHPQIDPKLLNKLHEYFHEPNKKFFELVGRTFDWH

SEQ ID NO: 16 - protein, 320 aa, Gallus gallus
MAAFLLGAVLLIVQPQIVPSRPAINSKAETSAQSAQRELLKKTSQKNDFKENIHSNGSCRQLPQTIIIGVRKGGTRALLEMLSLHPDIAAAESEVHFFDWEDHYRNGLQWYINQMPFSYPHQITVEKTPAYFTSPEVPERVYNMNQSMRLLLILRDPSERVLSDYTQVFYNHMQKHKPYPSIEQFLIKDGELNVDYKAINRSLYYIHMQNWLKYFPLDHIHIVDGDKLIKDPFPEIEKVERFLKLSPQINASNFYFNKTKGFYCLRDSGRERCLHESKGRAHPQVDTRLLEKLHEYFYEPNKKFFELVGRTFDWHSSVAS

SEQ ID NO: 17 - protein, 617 aa, Homo sapiens
MRCLAARVNYKTLIIICALFTLVTVLLWNKCSSDKAIQFPRRSSSGFRVDGFEKRAAASESNNYMNHVAKQQSEEAFPQEQQKAPPVVGGFNSNVGSKVLGLKYEEIDCLINDEHTIKGRREGNEVFLPFTWVEKYFDVYGKVVQYDGYDRFEFSHSYSKVYAQRAPYHPDGVFMSFEGYNVEVRDRVKCISGVEGVPLSTQWGPQGYFYPIQIAQYGLSHYSKNLTEKPPHIEVYETAEDRDKNKPNDWTVPKGCFMANVADKSRFTNVKQFIAPETSEGVSLQLGNTKDFIISFDLKFLTNGSVSVVLETTEKNQLFTIHYVSNAQLIAFKERDIYYGIGPRTSWSTVTRDLVTDLRKGVGLSNTKAVKPTKIMPKKVVRLIAKGKGFLDNITISTTAHMAAFFAASDWLVRNQDEKGGWPIMVTRKLGEGFKSLEPGWYSAMAQGQAISTLVRAYLLTKDHIFLNSALRATAPYKFLSEQHGVKAVFMNKHDWYEEYPTTPSSFVLNGFMYSLIGLYDLKETAGEKLGKEARSLYERGMESLKAMLPLYDTGSGTIYDLRHFMLGIAPNLARWDYHTTHINQLQLLSTIDESPVFKEFVKRWKSYLKGSRAKHN

SEQ ID NO: 18 - protein, 526 aa, Homo sapiens
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 19 - protein, 213 aa, synthetic construct
ASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRG

SEQ ID NO: 20 - protein, 592 aa, Homo sapiens
MSDSGSYGQSGGEQQSYSTYGNPGSQGYGQASQSYSGYGQTTDSSYGQNYSGYSSYGQSQSGYSQSYGGYENQKQSSYSQQPYNNQGQQQNMESSGSQGGRAPSYDQPDYGQQDSYDQQSGYDQHQGSYDEQSNYDQQHDSYSQNQQSYHSQRENYSHHTQDDRRDVSRYGEDNRGYGGSQGGGRGRGGYDKDGRGPMTGSSGGDRGGFKNFGGHRDYGPRTDADSESDNSDNNTIFVQGLGEGVSTDQVGEFFKQIGIIKTNKKTGKPMINLYTDKDTGKPKGEATVSFDDPPSAKAAIDWFDGKEFHGNIIKVSFATRRPEFMRGGGSGGGRRGRGGYRGRGGFQGRGGDPKSGDWVCPNPSCGNMNFARRNSCNQCNEPRPEDSRPSGGDFRGRGYGGERGYRGRGGRGGDRGGYGGDRSGGGYGGDRSSGGGYSGDRSGGGYGGDRSGGGYGGDRGGGYGGDRGGGYGGDRGGGYGGDRGGYGGDRGGGYGGDRGGYGGDRGGYGGDRGGYGGDRGGYGGDRSRGGYGGDRGGGSGYGGDRSGGYGGDRSGGGYGGDRGGGYGGDRGGYGGKMGGRNDYRNDQRNRPY

SEQ ID NO: 21 - protein, 208 aa, synthetic construct
MSDSGSYGQSGGEQQSYSTYGNPGSQGYGQASQSYSGYGQTTDSSYGQNYSGYSSYGQSQSGYSQSYGGYENQKQSSYSQQPYNNQGQQQNMESSGSQGGRAPSYDQPDYGQQDSYDQQSGYDQHQGSYDEQSNYDQQHDSYSQNQQSYHSQRENYSHHTQDDRRDVSRYGEDNRGYGGSQGGGRGRGGYDKDGRGPMTGSSGGDRGG

SEQ ID NO: 22 - protein, 708 aa, Caenorhabditis elegans
MESNQSNNGGSGNAALNRGGRYVPPHLRGGDGGAAAAASAGGDDRRGGAGGGGYRRGGGNSGGGGGGGYDRGYNDNRDDRDNRGGSGGYGRDRNYEDRGYNGGGGGGGNRGYNNNRGGGGGGYNRQDRGDGGSSNFSRGGYNNRDEGSDNRGSGRSYNNDRRDNGGDGQNTRWNNLDAPPSRGTSKWENRGARDERIEQELFSGQLSGINFDKYEEIPVEATGDDVPQPISLFSDLSLHEWIEENIKTAGYDRPTPVQKYSIPALQGGRDLMSCAQTGSGKTAAFLVPLVNAILQDGPDAVHRSVTSSGGRKKQYPSALVLSPTRELSLQIFNESRKFAYRTPITSALLYGGRENYKDQIHKLRLGCHILIATPGRLIDVMDQGLIGMEGCRYLVLDEADRMLDMGFEPQIRQIVECNRMPSKEERITAMFSATFPKEIQLLAQDFLKENYVFLAVGRVGSTSENIMQKIVWVEEDEKRSYLMDLLDATGDSSLTLVFVETKRGASDLAYYLNRQNYEVVTIHGDLKQFEREKHLDLFRTGTAPILVATAVAARGLDIPNVKHVINYDLPSDVDEYVHRIGRTGRVGNVGLATSFFNDKNRNIARELMDLIVEANQELPDWLEGMSGDMRSGGGYRGRGGRGNGQRFGGRDHRYQGGSGNGGGGNGGGGGFGGGGQRSGGGGGFQSGGGGGRQQQQQQRAQPQQDWWS

SEQ ID NO: 23 - protein, 169 aa, Synthetic construct
FMESNQSNNGGSGNAALNRGGRYVPPHLRGGDGGAAAAASAGGDDRRGGAGGGGYRRGGGNSGGGGGGGYDRGYNDNRDDRDNRGGSGGYGRDRNYEDRGYNGGGGGGGNRGYNNNRGGGGGGYNRQDRGDGGSSNFSRGGYNNRDEGSDNRGSGRSYNNDRRDNGGDG

SEQ ID NO: 24 - protein, 724 aa, Homo sapiens
MGDEDWEAEINPHMSSYVPIFEKDRYSGENGDNFNRTPASSSEMDDGPSRRDHFMKSGFASGRNFGNRDAGECNKRDNTSTMGGFGVGKSFGNRGFSNSRFEDGDSSGFWRESSNDCEDNPTRNRGFSKRGGYRDGNNSEASGPYRRGGRGSFRGCRGGFGLGSPNNDLDPDECMQRTGGLFGSRRPVLSGTGNGDTSQSRSGSGSERGGYKGLNEEVITGSGKNSWKSEAEGGESSDTQGPKVTYIPPPPPEDEDSIFAHYQTGINFDKYDTILVEVSGHDAPPAILTFEEANLCQTLNNNIAKAGYTKLTPVQKYSIPIILAGRDLMACAQTGSGKTAAFLLPILAHMMHDGITASRFKELQEPECIIVAPTRELVNQIYLEARKFSFGTCVRAVVIYGGTQLGHSIRQIVQGCNILCATPGRLMDIIGKEKIGLKQIKYLVLDEADRMLDMGFGPEMKKLISCPGMPSKEQRQTLMFSATFPEEIQRLAAEFLKSNYLFVAVGQVGGACRDVQQTVLQVGQFSKREKLVEILRNIGDERTMVFVETKKKADFIATFLCQEKISTTSIHGDREQREREQALGDFRFGKCPVLVATSVAARGLDIENVQHVINFDLPSTIDEYVHRIGRTGRCGNTGRAISFFDLESDNHLAQPLVKVLTDAQQDVPAWLEEIAFSTYIPGFSGSTRGNVFASVDTRKGKSTLNTAGFSSSQAPNPVDDESWD

SEQ ID NO: 25 - protein, 386 aa, Homo sapiens
MEDEMPKTLYVGNLSRDVTEALILQLFSQIGPCKNCKMIMDTAGNDPYCFVEFHEHRHAAAALAAMNGRKIMGKEVKVNWATTPSSQKKDTSSSTVVSTQRSQDHFHVFVGDLSPEITTEDIKAAFAPFGRISDARVVKDMATGKSKGYGFVSFFNKWDAENAIQQMGGQWLGGRQIRTNWATRKPPAPKSTYESNTKQLSYDEVVNQSSPSNCTVYCGGVTSGLTEQLMRQTFSPFGQIMEIRVFPDKGYSFVRFNSHESAAHAIVSVNGTTIEGHVVKCYWGKETLDMINPVQQQNQIGYPQPYGQWGQWYGNAQQIGQYMPNGWQVPAYGMYGQAWNQQGFNQTQSSAPWMGPNYGVQPPQGQNGSMLPNQPSGYRVAGYETQ

SEQ ID NO: 26 - protein, 5 aa, synthetic construct
  VAR_SEQ 4: note = X is any amino acid residue other than proline
VPGXG

SEQ ID NO: 27 - protein, 992 aa, Mus musculus
MKGLSGSRSHHHGITCEAACDSLSHHSDHKPYLLSPVDHHPADHPYYTQRNSFQAECVGPFSDPLASSTFPRRHYTSQQELKDESALVPRTLATKANRLPTNLLDQFERQLPLSRDGYHTLQYKRTAVEHRSDSPGRIRHLVHSVQKLFTKSHSLEGPSKGSVNGGKASPDESQTLRYGKRSKSKERRSESKARSNASNASPTSPSWWSSDDNLDGDMCLYHTPSGVMTMGRCPDRSASQYFMEAYNTISEQAVKASRSNNDIKCSTCANLPVTLDAPLLKKSAWSSTLTVSRAREVYQKASVNMDQAMVKSEACQQERSCQYLQVPQDEWSGYTPRGKDDEIPCRRMRSGSYIKAMGDEDSGDSDTSPKPSPKVAARRESYLKATQPSLTELTTLKISNEHSPKLQIRSHSYLRAVSEVSINRSLDSLDPAGLLTSPKFRSRNESYMRAMSTISQVSEMEVNGQFESVCESVFSELESQAVEALDLPLPGCFRMRSHSYVRAIEKGCSQDDECVSLRSSSPPRTTTTVRTIQSSTGVIKLSSAVEVSSCITTYKKTPPPVPPRTTTKPFISITAQSSTESAQDAYMDGQGQRGDMISQSGLSNSTESLDSMKALTAAIEAANAQIHGPASQHMGSNAAAVTTTTTIATVTTEDRKKDFKKNRCLSIGIQVDDAEEPEKMAESKTSNKFQSVGVQVEEEKCFRRFTRSNSVTTAVQADLDFHDNLENSLESIEDNSCPGPMARQFSRDASTSTVSIQGSGNHYHACAADDDFDTDFDPSILPPPDPWIDSITEDPLEAVQRSVCHRDGHWFLKLLQAERDRMEGWCKLMEREERENNLPEDILGKIRTAVGSAQLLMAQKFYQFRELCEENLNPNAHPRPTSQDLAGFWDMLQLSIENISMKFDELHQLKANNWKQMDPLDKKERRAPPPVPKKPAKGPAPLIRERSLESSQRQEARKRLMAAKRAASVRQNSATESAESIEIYIPEAQTRL

SEQ ID NO: 28 - protein, 172 aa, synthetic construct
MQDEWSGYTPRGKDDEIPCRRMRSGSYIKAMGDEDSGDSDTSPKPSPKVAARRESYLKATQPSLTELTTLKISNEHSPKLQIRSHSYLRAVSEVSQMDPLDKKERRAPPPVPKKPAKGPAPLIRERSLESSQRQEARKRLMAAKRAASVRQNSATESAESIEIYIPEAQTRL

SEQ ID NO: 29 - protein, 361 aa, Homo sapiens
MSTAREQPIFSTRAHVFQIDPATKRNWIPAGKHALTVSYFYDATRNVYRIISIGGAKAIINSTVTPNMTFTKTSQKFGQWADSRANTVYGLGFASEQHLTQFAEKFQEVKEAARLAREKSQDGGELTSPALGLASHQVPPSPLVSANGPGEEKLFRSQSADAPGPTERERLKKMLSEGSVGEVQWEAEFFALQDSNNKLAGALREANAAAAQWRQQLEAQRAEAERLRQRVAELEAQAASEVTPTGEKEGLGQGQSLEQLEALVQTKDQEIQTLKSQTGGPREALEAAEREETQQKVQDLETRNAELEHQLRAMERSLEEARAERERARAEVGRAAQLLDVSLFELSELREGLARLAEAAP

SEQ ID NO: 30 - protein, 1730 aa, Mus musculus
MDGPGASAVVVRVGIPDLQQTKCLRLDPTAPVWAAKQRVLCALNHSLQDALNYGLFQPPSRGRAGKFLDEERLLQDYPPNLDTPLPYLEFRYKRRVYAQNLIDDKQFAKLHTKANLKKFMDYVQLHSTDKVARLLDKGLDPNFHDPDSGECPLSLAAQLDNATDLLKVLRNGGAHLDFRTRDGLTAVHCATRQRNAGALTTLLDLGASPDYKDSRGLTPLYHSALGGGDALCCELLLHDHAQLGTTDENGWQEIHQACRFGHVQHLEHLLFYGANMGAQNASGNTALHICALYNQESCARVLLFRGANKDVRNYNSQTAFQVAIIAGNFELAEVIKTHKDSDVVPFRETPSYAKRRRLAGPSGLASPRPLQRSASDINLKGDQPAASPGPTLRSLPHQLLLQRLQEEKDRDRDGELENDISGPSAGRGGHNKISPSGPGGSGPAPGPGPASPAPPAPPPRGPKRKLYSAVPGRKFIAVKAHSPQGEGEIPLHRGEAVKVLSIGEGGFWEGTVKGRTGWFPADCVEEVQMRQYDTRHETREDRTKRLFRHYTVGSYDSLTSHSDYVIDDKVAILQKRDHEGFGFVLRGAKAETPIEEFTPTPAFPALQYLESVDVEGVAWRAGLRTGDFLIEVNGVNVVKVGHKQVVGLIRQGGNRLVMKVVSVTRKPEEDGARRRAPPPPKRAPSTTLTLRSKSMTAELEELASIRRRKGEKLDEILAVAAEPTLRPDIADADSRAATVKQRPTSRRITPAEISSLFERQGLPGPEKLPGSLRKGIPRTKSVGEDEKLASLLEGRFPRSTSMQDTVREGRGIPPPPQTAPPPPPAPYYFDSGPPPTFSPPPPPGRAYDTVRSSFKPGLEARLGAGAAGLYDPSTPLGPLPYPERQKRARSMIILQDSAPEVGDVPRPAPAATPPERPKRRPRPSGPDSPYANLGAFSASLFAPSKPQRRKSPLVKQLQVEDAQERAALAVGSPGPVGGSFAREPSPTHRGPRPGSLDYSSGEGLGLTFGGPSPGPVKERRLEERRRSTVFLSVGAIEGSPPSADLPSLQPSRSIDERLLGTGATTGRDLLLPSPVSALKPLVGGPSLGPSGSTFIHPLTGKPLDPSSPLALALAARERALASQTPSRSPTPVHSPDADRPGPLFVDVQTRDSERGPLASPAFSPRSPAWIPVPARREAEKPPREERKSPEDKKSMILSVLDTSLQRPAGLIVVHATSNGQEPSRLGAEEERPGTPELAPAPMQAAAVAEPMPSPRAQPPGSIPADPGPGQGSSEEEPELVFAVNLPPAQLSSSDEETREELARIGLVPPPEEFANGILLTTPPPGPGPLPTTVPSPASGKPSSELPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASTGLASAAGPARPRYLFQRRSKLWGDPVESRGLPGPEDDKPTVISELSSRLQQLNKDTRSLGEEPVGGLGSLLDPAKKSPIAAARLFSSLGELSTISAQRSPGGPGGGASYSVRPSGRYPVARRAPSPVKPASLERVEGLGAGVGGAGRPFGLTPPTILKSSSLSIPHEPKEVRFVVRSVSARSRSPSPSPLPSPSPGSGPSAGPRRPFQQKPLQLWSKFDVGDWLESIHLGEHRDRFEDHEIEGAHLPALTKEDFVELGVTRVGHRMNIERALRQLDGS

SEQ ID NO: 31 - protein, 268 aa, synthetic construct
MDTRHETREDRTKRLFRHYTVGSYDSLTSHSDYVIDDKVAILQKRDHEGFGFVLRGAKAETPIEEFTPTPAFPALQYLESVDVEGVAWRAGLRTGDFLIEVNGVNVVKVGHKQVVGLIRQGGNRLVMKVVSVTRDEETREELARIGLVPPPEEFANGILLTTPPADGHTFLLEKPPVPPKPKLKSPLGKGPGPRRPFQQKPLQLWSKFDVGDWLESIHLGEHRDRFEDHEIEGAHLPALTKEDFVELGVTRVGHRMNIERALRQLDGS

SEQ ID NO: 32 - protein, 18 aa, synthetic construct
LEQYANQLADQIIKEATE

SEQ ID NO: 33 - protein, 49 aa, synthetic construct
LRECELYVQKHNIQALLKDSIVQLCTARPERPMAFLREYFERLEKEEAK

SEQ ID NO: 34 - protein, 183 aa, Streptomyces avidinii
MRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 35 - protein, 291 aa, Rattus norvegicus
MEFSRPPLVHVKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 36 - protein, 101 aa, Saccharomyces cerevisiae
MSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATY

(19) United States
(12) Patent Application Publication
(10) Pub. No.: US 2024/0401096 A1
(43) Pub. Date: Dec. 5, 2024

(54) BIOMOLECULAR CONDENSATE MANUFACTURING SCAFFOLD

(71) Applicant: Celvant LLC, Cincinnati, OH (US)

(72) Inventors: JAMES ROTHMAN, Westhampton, NY (US); JOHN BALDONI, NAPLES, FL (US); NATHANIEL S. SCHOCKER, PEARLAND, TX (US); DANIEL H. LAJINESS, FAIRFIELD, OH (US)

(21) Appl. No.: 18/679,102

(22) Filed: May 30, 2024

Related U.S. Application Data
(60) Provisional application No. 63/469,673, filed on May 30, 2023.
(60) Provisional application No. 63/645,458, filed on May 10, 2024.

(51) Int. Cl. C12P 19/04; C12N 9/10; C12N 9/90; C12N 11/18

(52) U.S. Cl. CPC C12P 19/04; C12N 9/13; C12N 9/90; C12N 11/18; C12Y 208/02; C12Y 208/02029; C12Y 208/0203; C12Y 501/03

(57) ABSTRACT

Methods and systems for synthesizing biological products within biomimetic condensates that enhance the concentration of enzymes, substrates, co-factors, and other molecules involved in the synthesis. The one or more enzymes involved in catalysis can be engineered to comprise low-complexity amino acid sequences or phase separation domains that can be controlled to drive reversible liquid-liquid phase separation, wherein the resulting biomimetic condensates comprise a traversable phase boundary between their dense internal portion and the less-crowded aqueous composition within which the condensates are maintained. Alternatively, biomimetic condensates can be first formed within the aqueous composition from affinity-tagged scaffold proteins that do not take part in catalysis, but are capable of recruiting into the condensate catalytic enzymes fused to an affinity tag partner. One or more enzymes can comprise or be recruited inside of the biomimetic condensate to generate a wide variety of desired chemical products.

### CROSS-REFERENCE TO RELATED APPLICATIONS

The instant application claims of the benefit of U.S. Provisional Applications 63/469,673, filed on May 30, 2023, and 63/645,458, filed May 10, 2024, the disclosures of which are hereby incorporated by reference in their entireties.

### FIELD OF THE INVENTION

The present invention relates to methods for forming and using biomolecular condensates as manufacturing scaffolds for the in vitro synthesis of biochemical products.

### REFERENCE TO SEQUENCE LISTING

The present application is being filed along with a sequence listing in electronic format. The sequence listing is provided as a file entitled “CEL-001M_Sequence_Listing.xml” created on May 30, 2024 and which is 46,768 bytes in size. The information in electronic format of the sequence listing is incorporated by reference in its entirety.

### BACKGROUND OF THE INVENTION

Most active pharmaceutical ingredients (API) are currently manufactured through the sequential build of an organic molecule through a step-wise series of chemical reactions. Biosynthetic chemistry has emerged as an alternative to traditional organic synthesis to take advantage of enzymatic specificity, catalytic efficiency, and green chemistry. Currently, traditional in vitro processes involve the mixing in solution of all the enzymes, reagents, and cofactors required to make the desired product. At a laboratory or industry scale, the rate of product formation in biosynthetic chemistry is dictated by the concentrations of biomolecules and dependent on the stochastic interactions between each of the components, which are in constant motion within the solution. As the scale gets larger and larger, bulk diffusion and mixing of components become primary determinants of product formation rate. As a result, the cost of materials required to make large quantities of product in an uncontrolled bioreactor is often prohibitive.

Bulk diffusion is a phenomenon not only exhibited in in vitro syntheses, but within the cell as well. In simple bacteria (˜1 μm size), the internal environment has a small enough volume that a vast majority of the small molecular synthesis takes place within the dispersed cytoplasm. However, when much larger eukaryotic (nucleated) cells (˜10 to 30 μm in size, and 10³ to 10⁴ the volume of bacteria) evolved, the diffusion of reaction components emerged as a determinant of reaction rate. It is fundamental that products can only be formed when enzymes encounter their substrates, and as the volume of the system increases, the frequency of encounters by simple diffusion decreases. Eukaryotic cells solve this problem by organizing internally into numerous bacterial-size subcellular compartments within which related enzymes and their substrates can tightly congregate. Accordingly, even when the overall concentration of a given enzyme may be low, the rate of product formation has been demonstrated to be 10- to 100-times higher when condensed into a compartment.

The primary compartments within eukaryotic cells are organelles. Organelles were discovered in the 1950s to comprise lipid bilayer-containing membranes that retain the enzymes inside of them, but also possess special systems to select and import their enzyme content from the surrounding cytoplasm, which houses the ribosomes that make them. Exemplary organelles include the endoplasmic reticulum, Golgi apparatus, lysosomes, mitochondria, and others. However, attempts at compartmentalized biosynthesis based on membrane-organelles are generally futile, because enclosing the enzymes efficiently in vitro is extremely difficult, as is transporting substrates and products freely into and out of the compartment.

Consequently, there is a need to develop a manufacturing scaffold in which substrates, co-factors, enzymes, and/or other reagents can be confined into a relatively limited space, regardless of the volume of the reaction vessel, to increase the speed and efficiency of the synthesis of biochemical products and promote the free transfer of substrates and products.

### SUMMARY OF THE INVENTION

The present invention provides compositions, systems, and methods for synthesizing biologically active materials within one or more biomimetic condensates. Particularly, the condensates can place into proximity a vast array of reagents, substrates, buffers, cofactors, protein and nucleotide receptors, enzymes and other molecules that would otherwise diffuse throughout the entirety of an aqueous composition in vitro, and facilitate the efficient biosynthesis of compounds that typically are only achievable through chemical means.

In an aspect of the invention, a method for catalyzing the in vitro enzymatic synthesis of a biological product within a biomimetic condensate comprises the steps of: (a) providing an aqueous composition comprising one or more enzymes, the one or more enzymes having a biological activity, wherein the biological activity consists of producing a biological product upon reacting with one or more substrates; (b) assembling the one or more enzymes into a biomimetic condensate having an internal portion and an external portion separated by a phase boundary; (c) introducing one or more substrates into the aqueous composition; and (d) initiating a chemical reaction between at least one of the enzymes and at least one of the substrates to synthesize the biological product.

In various embodiments, any of the enzymes can comprise one or more low-complexity amino acid sequences, which together can comprise a phase separation domain. Non-limiting examples of such phase separation domains are intrinsically disordered regions (IDR) and elastin-like polypeptide (ELP) domains. In response to one or more changes in the chemical or physical properties of the composition, non-limiting examples of which are composition pH, temperature, volume, pressure, salt concentration, and the presence of one or more crowding agents (non-limiting examples of which are polyethylene glycol and dextran), the phase separation domains of multiple protein molecules diffusing within the aqueous composition can spontaneously arrange themselves into an ordered structure, leading to the formation of a liquid condensate.

In various embodiments, adding the one or more enzymes into the aqueous composition can also initiate their self-assembly into condensates so long as the enzyme concentration within the aqueous is above a defined threshold. Non-limiting examples of such concentration thresholds are greater than about 100 nM, 500 nM, 1 μM, 5 μM, 10 μM, 20 μM, or about 50 μM. Although a phase boundary separates the denser internal portion of the condensate and the less dense aqueous composition at the external portion of the condensate, both portions of the condensate are in a liquid phase. Accordingly, any of the biomimetic condensates within an aqueous composition that also have a liquid internal phase can be referred to as “liquid-liquid phase-separated (LLPS) droplets.”

In various embodiments, methods of the present invention can comprise a single enzyme having one or more low-complexity amino acid sequences and/or one or more phase separation domains. Upon assembling into LLPS droplets, the addition into the aqueous composition of one or more substrates capable of reacting with the enzyme can initiate synthesis of the biological product. Without being limited by a particular theory, it is believed that because the internal portion and the external portion of the biomimetic condensates are both liquids and the phase boundary is not formed from an impermeable or semi-permeable membrane, substrates and products can generally freely traverse in and out of the condensate.

In various embodiments, the one or more enzymes comprises a plurality of enzymes having a concerted biological activity, in which the biological activity of a first enzyme produces an intermediate product utilized as a starting material for a second enzyme to synthesize a second product. Those of ordinary skill in the art would understand that two, three, four, or more enzymes can all have a concerted biological activity in which the biological activity of one enzyme synthesizes a starting material for another enzyme, and so on, until the final biological product is synthesized. In one non-limiting example, lidocaine can be synthesized by the action of two enzymes working in concert, alcohol dehydrogenase and lipase B. In another non-limiting example, higher order heparan sulfates and/or heparin can be synthesized by the concerted biological activity of two, three, or four enzymes. Details regarding the synthesis of both classes of compounds are provided in further detail, below.

In various embodiments, a plurality of enzymes having a concerted biological activity can assemble within the same condensate, and substrates for each enzyme can either be added to the aqueous composition simultaneously or sequentially. Further, in some embodiments, each of the enzymes utilized to form a biological product can comprise a low-complexity amino acid sequence and/or phase separation domain. In other embodiments, fewer than all the enzymes within a condensate comprise a low-complexity amino acid sequence and/or phase separation domain. Without being limited by a particular theory, it is believed that enzymes lacking such sequences or domains can nonetheless be recruited inside of the biomimetic condensates based on their inherent binding affinity with one or more other enzymes. As a non-limiting example, a three-enzyme system for forming a biological product can be utilized in which only one, or two, of the enzymes comprise a low-complexity amino acid sequence and/or phase separation domain.

In various embodiments, a plurality of enzymes having a concerted biological activity can assemble in multiple condensates that abut each other, in which the internal portion of each condensate has a different density relative to each other and to the aqueous composition. In a non-limiting example and in some embodiments, such multi-condensate systems can be formed by first engineering a first low-complexity sequence or phase separation domain into a first enzyme, followed by engineering a second low-complexity sequence or phase separation domain into a second enzyme. Mixing the two enzymes into the same aqueous composition causes the formation of a first set of condensates comprising the first enzyme and a second set of condensates comprising the second enzyme, wherein a plurality of condensates within the first set of condensates abuts a plurality of condensates within the second set of condensates. Without being limited by a particular theory, it is believed that any added or synthesized substrates, intermediates, and/or products can move freely among and through the first set of condensates, the second set of condensates, and the aqueous composition, and that the formation of adjacent condensates limits intermediates from randomly diffusing throughout the aqueous composition.

In various embodiments, the phase separation domain within any of the enzymes is a synthetic or natural IDR. For natural IDR's particularly, the IDR can encompass the entire amino acid sequence of a structurally-undefined protein, or otherwise comprise a subset of low-complexity, structurally-undefined amino acids with a protein that otherwise possess a secondary and/or tertiary structure. Generally, IDRs are deficient in hydrophobic amino acids and instead typically comprise a limited variety of polar, charged, and aromatic amino acids, and often have high quantities of serine, glutamine, and glycine residues.

In various embodiments, the amino acid sequence for any catalytic enzyme can be fused to any IDR sequence. Multiple databases compiling characteristic sequences, properties, and functions of IDRs are well-known in the art, and are updated regularly as new IDRs in nature are elucidated. Further, multiple prediction algorithms and applications are also available for those of ordinary skill to design their own IDR sequences.

As non-limiting example, an IDR fused to any of the enzymes described herein can comprise at least a fragment of a natural IDR present within the amino acid sequence of an enzyme selected from the group consisting of fused in sarcoma (FUS) protein, TATA-box binding protein associated factor 15 (TAF), P-granule protein LAF-1 (LAF), Ddx4 helicase (DDX), and Tia1 cytotoxic granule-associated RNA binding protein (TIA). In further embodiments, an IDR based on FUS, TAF, LAF, DDX, or TIA can comprise the amino acid sequence of SEQ ID NO: 18, SEQ ID NO: 20, SEQ ID NO: 22, SEQ ID NO: 24, and SEQ ID NO: 25, respectively. In even further embodiments, one or more of the enzymes can be fused to an FUS IDR having the amino acid sequence of SEQ ID NO: 19. In even further embodiments, one or more of the enzymes can be fused to a TAF IDR having the amino acid sequence of SEQ ID NO: 21. In even further embodiments, one or more of the enzymes can be fused to a LAF IDR having the amino acid sequence of SEQ ID NO: 23.

In various embodiments, the phase separation domain within any of the enzymes is an ELP motif, comprising oligomeric repeats of the pentapeptide Val-Pro-Gly-Xaa-Gly, wherein Xaa can be any amino acid except proline (SEQ ID NO: 26). Any ELP motif, including but not limited to an ELP motifs having one or more repeats of SEQ ID NO: 26, can be fused at either the N- or C-terminus of an enzyme of interest. Fusion proteins comprising an enzyme of interest and an ELI are described in detail in U.S. Pat. Nos. 6.852,834 and 10,526,396, the disclosures of which are incorporated by reference in their entireties. Particularly, and in various embodiments, ELP motifs can undergo reversible inverse temperature transitions, where they are highly soluble in water below the inverse transition temperature Tₜ, but undergo a sharp phase transition over 2-3° C. beyond the Tₜ.

In another aspect of the invention, the biosynthesis of biological products can be conducted inside biomimetic condensates that are pre-formed within an aqueous composition prior to adding one or more enzymes that take part in the catalysis. Accordingly, and in some embodiments, a method for the in vitro enzymatic synthesis of a biological product within a biomimetic condensate can comprise the steps of; (a) providing an aqueous composition comprising one or more scaffold proteins, wherein at least one of the scaffold proteins is fused to an affinity tag; (b) assembling the one or more scaffold proteins into a biomimetic condensate having an internal portion and an external portion separated by a phase boundary; (c) introducing one or more enzymes into the aqueous composition, wherein at least one of the enzymes is fused to affinity tag partner; (d) binding the affinity tag partner with the affinity tag, thereby recruiting the one or more enzymes into the biomimetic condensate; (e) introducing one or more substrates into the aqueous composition; and (f) initiating a chemical reaction between at least one of the enzymes and at least one of the substrates to synthesize the biological product.

In various embodiments, enzymes taking part in the chemical reaction are not fused to a low-complexity amino acid sequence and/or phase transition domain. As a non-limiting example, the one or more scaffold proteins can comprise at least a fragment of a GKAP protein, at least a fragment of a Homer protein, and at least a fragment of a Shank protein, which are well-known in the art to be the most abundant proteins within a multivalent interaction network formed by major excitatory postsynaptic density (PSD) proteins within neurological synapses. Particularly, GKAP, Shank, and Homer collectively have been shown to self-assemble into LLPS protein droplets both in vivo and in vitro. In various embodiments. GKAP, Homer, and Shank utilized in accordance with methods of the present invention can comprise the amino acid sequences of SEQ ID NO: 27, SEQ ID NO: 29, and SEQ ID NO: 30, respectively, which can spontaneously form LLPS droplets in vitro when mixed in an aqueous composition at concentrations of at least 1 μM each, and particularly at least 5 μM each. In various embodiments, GKAP and Shank can be truncated, comprising the amino acid sequences of SEQ ID NO: 28 and 31, respectively.

In various embodiments, one or more of GKAP, Homer, and Shank can be fused to a polypeptide-based affinity tag that has high affinity with another polypeptide, referred to herein as an “affinity tag partner.” Non-limiting examples of affinity tag-affinity tag partners useful in accordance with methods of the present invention are the RIDD and RIAD peptides, as well as streptavidin and biotin. Techniques for fusing RIDD and RIAD to an enzyme of interest are well-known in the art. In various embodiments, the amino acid sequence of RIDD is SEQ ID NO: 33. In various embodiments, the amino acid sequence of RIAD is SEQ ID NO: 32. In various embodiments, the amino acid sequence of Streptavidin is SEQ ID NO: 34.

In various embodiments, a RIAD peptide motif having the amino acid sequence of SEQ ID NO: 32 is fused to both Homer and Shank. Without being limited by a particular theory, it is believed that the tagging of Homer and Shank with the RIAD peptide motif has minimal to no impact on the ability of GKAP, Homer, and Shank to assemble into an LLPS droplet. In some embodiments, GKAP is truncated to comprise a minimum number of fragments to maintain its ability to interact with Homer and Shank to facilitate expression of soluble protein, possessing the amino acid sequence SEQ ID NO: 28. Similarly, and in other embodiments, RIAD-tagged Shank is truncated to comprise a minimum number of fragments to maintain its ability to interact with GKAP and Homer to facilitate expression of soluble protein, possessing the amino acid sequence SEQ ID NO: 31.

In various embodiments, the affinity tag partner that is fused to the one or more catalytic enzymes taking part in the chemical reaction is the RIDD peptide motif, comprising the amino acid sequence SEQ ID NO: 33. RIDD can be fused to either the N- or C-terminus of an enzyme of interest, based on the desired expression plasmid. Without being limited by a particular theory, it is believed that upon adding of RIDD-tagged enzymes to an aqueous composition in which a GKAP-Homer-Shank condensate scaffold has been pre-formed, the RIDD-tagged enzymes are able to traverse the phase boundary and accumulate within the LLPS droplets based on RIDD and RIAD's strong binding affinity (K_{d}=1 nM). In various embodiments, however, the equilibrium dissociation constant (K_{d}) can be less than 1 μM, 750 nM, 500 nM, 250 nM, 100 nM, 50 nM, 1 nM, 500 pM, or 1 pM.

As another non-limiting example, the affinity tag can be biotin. Techniques for biotinylating enzymes are well-known in the art. Further, biotin's K_{d} with streptavidin is 40 fM (4×10⁻¹⁴ M), an interaction that is more than 10,000× stronger than RIDD and RIAD, promoting an even more rapid recruitment of streptavidin-tagged enzymes of interest into the condensate. In a non-limiting example, a streptavidin tag can comprise at least a fragment of the amino acid sequence SEQ ID NO: 34.

In another aspect of the invention, a pre-formed condensate scaffold can be formed using a combination of IDR fusions in conjunction with a biotin-streptavidin affinity pair. In various embodiments, a condensate scaffold can first be formed by biotinylating the IDR of FUS and assembling a cluster in an aqueous solution upon the addition of streptavidin. The interaction of biotinylated IDRs with streptavidin is known to rapidly and stably tetramerize the IDR and form generally-spherical protein droplets. To minimize potential aggregation, and in some embodiments, the small and highly-soluble SUMO protein domain can also be fused to the biotinylated FUS IDRs. In some embodiments, TAF or LAF can be biotinylated instead of FUS. In further embodiments, the aqueous composition can further comprise a crowding agent, one non-limiting example of which is polyethylene glycol, and particularly, 15% polyethylene glycol.

Once the biotinylated-IDR-streptavidin condensates (with or without SUMO) are formed in solution, IDR-tagged enzymes of interest can be recruited inside the condensate upon their addition to the aqueous composition. In various embodiments, any IDR sequence can be fused to any enzyme of interest. In further embodiments, any of the FUS, TAF, LAF, DDX, or TIA (SEQ ID NO: 18, SEQ ID NO: 20, SEQ ID NO: 22, SEQ ID NO: 24, and SEQ ID NO: 25, respectively) IDRs can be fused to an enzyme of interest. In even further embodiments, FUS, TAF, and LAF can comprise the amino acid sequences of SEQ ID NO: 19. SEQ ID NO: 21, or SEQ ID NO: 23, respectively.

In various embodiments and in conjunction with any of the methods for forming condensates described above, the local concentration of catalytic enzymes within the condensates is increased relative to the concentration of the enzymes within the entire aqueous composition. In various embodiments, enzyme concentration within a condensate can be enriched at least 5, 10, 15, 20, 25, 30, 40, 50, 60, 70, 80, 90, 100, 125, 150, 175, or 200 times, up to at least 250 times. In various embodiments, the concentration of substrates, co-factors, and other materials within the condensate can similarly be enriched at least 5, 10, 15, 20, 25, 30, 40, 50, 60, 70, 80, 90, 100, 125, 150, 175, or 200 times, up to at least 250 times. In various embodiments, the enrichment of both enzymes and substrates within the condensates can approximate or replicate the conditions exhibited for synthesizing biological products inside the cell, leading to catalytic efficiencies and product yields that are comparable to those observed in vivo.

A non-limiting example of an in vitro reaction that can be made more efficient by conducting the reaction within a biomimetic condensate is the biological activity of sulfotransferases to transfer a sulfo group from 3′-phosphoadenosine 5′-phosphosulfate (PAPS) to heparosan-based polysaccharides to synthesize heparan sulfate. Of the four heparan sulfate sulfotransferases, three (2OST, 6OST, and 3OST) possess only the single function of sulfo group transfer, while NDST has a dual function of N-deacetylating and N-sulfating heparosan. NDST, 2OST, 6OST, and 3OST are members of Enzyme Class 2.8.2.8, 2.8.2.-, 2.8.2.-, and 2.8.2.23, respectively.

As eukaryotic sulfotransferases, those of ordinary skill in the art would appreciate that members of any of the above classes have been identified by their sequence, structure, and/or experimental observation to have biological activity with PAPS and a heparosan-based polysaccharide to synthesize heparan sulfate. Particularly, those of ordinary skill in the art would appreciate that the concerted in vivo biological activity of the four enzymes results in the synthesis of heparin. Accordingly, and in various embodiments, any active wild-type or engineered NDST, 2OST, 6OST, or 3OST amino acid sequence can be fused to an IDR, ELP, or affinity tag or affinity tag partner and incorporated into a biomimetic condensate. Non-limiting examples of such 2OST enzymes are SEQ ID NO: 8, SEQ ID NO: 9, and SEQ ID NO: 10. Non-limiting examples of such 6OST enzymes are SEQ ID NO: 11, SEQ ID NO: 12, and SEQ ID NO: 13. Non-limiting examples of such 3OST enzymes are SEQ ID NO: 14, SEQ ID NO: 15, and SEQ ID NO: 16. Countless other non-limiting examples of such heparan sulfate sulfotransferase enzymes are described in detail within U.S. Pat. Nos. 5,541,095, 5,817,487, 5,834,282, 6,861,254, 8,771,995, 9,951,149, 11,473,068, 11,542,534, 11,572,549, 11,572,550, 11,629,364, 11,692,180, 11,708,567, 11,708,593, 11,767,518, and 11,773,382, U.S. Pat Pub Nos. 2009/0035787, 2013/0296540, and 2016/0122446, as well as unpublished U.S. Provisional Patent No. 63/641,453, the disclosures of which are all incorporated by reference in their entireties.

In a further embodiment, condensates comprising a 20)S enzyme can be engineered to also comprise a glucuronyl C₅-epimerase (hereinafter, “Epi”), particularly for reactions in which the sulfo group acceptor is un-epimerized N-sulfated heparan sulfate (N-HS), which contains only hexuronyl residues in their glucuronic acid form. Non-limiting examples of such Epi enzymes are SEQ ID NO: 17. In various embodiments, Epi can be expressed and purified as a single enzyme unit. In other various embodiments, Epi can be expressed and purified as a fusion with 2OST, facilitating the formation of a complex between the two enzymes.

Accordingly, in various embodiments, a method for catalyzing the in vitro enzymatic synthesis of heparan sulfate within a biomimetic condensate can comprise the following steps: (a) providing an aqueous composition comprising one or more heparan sulfate sulfotransferases selected from the group consisting of hexuronyl 2-O sulfotransferase (2OST), glucosaminyl 6-O sulfotransferase (6OST), glucosaminyl 3-O sulfotransferase (3OST), and any combination thereof, the one or more heparan sulfate sulfotransferases having a biological activity, wherein the biological activity consists of producing heparan sulfate upon reacting with PAPS and a heparosan-based polysaccharide, wherein at least one of the heparan sulfate sulfotransferases is a fusion protein comprising the sulfotransferase fused to a low-complexity phase separation domain selected from the group consisting of an intrinsically disordered region (IDR) and an elastin-like polypeptide (ELP) domain; (b) assembling the one or more heparan sulfate sulfotransferases into a biomimetic condensate having an internal portion and an external portion separated by a phase boundary, wherein the one or more heparan sulfate sulfotransferases are contained within the internal portion, and wherein the internal portion is a liquid; (c) introducing one or more substrates into the aqueous composition, wherein the one or more substrates can freely traverse the phase boundary between the external portion and internal portion of the biomimetic condensate, and wherein the one or more substrates comprise PAPS and a sulfo group acceptor selected from the group consisting of N-sulfated heparan sulfate (N-HS), N-,2-O-sulfated heparan sulfate (N2-HS), N-,2-O,6-O-sulfated heparan sulfate (N26-HS), and any combination thereof, and (d) initiating a chemical reaction between at least one of the heparan sulfate sulfotransferases, PAPS, and at least one of the sulfo group acceptors to synthesize the biological product. In further embodiments, the aqueous composition further comprises a glucuronyl C₅-epimerase enzyme. In even further embodiments, Epi comprises an IDR or an ELP domain. In various embodiments, when a 3OST enzyme is selected as one of the heparan sulfate sulfotransferases, the heparan sulfate product is heparin.

Additionally, and in various embodiments, a method for the in vitro enzymatic synthesis of heparan sulfate within a biomimetic condensate can comprise the steps of: (a) providing an aqueous composition comprising one or more scaffold proteins, the one or more scaffold proteins comprising at least a fragment of a GKAP protein, at least a fragment of a Homer protein, and at least a fragment of a Shank protein, wherein at least one of GKAP, Homer, Shank proteins is fused to an affinity tag; (b) assembling the one or more scaffold proteins into a biomimetic condensate having an internal portion and an external portion separated by a phase boundary, wherein the one or more scaffold proteins are contained within the internal portion, and wherein the internal portion is a liquid; (c) introducing one or more heparan sulfate sulfotransferases into the aqueous composition, wherein the one or more heparan sulfate sulfotransferases are selected from the group consisting of hexuronyl 2-O sulfotransferase (2OST), glucosaminyl 6-O sulfotransferase (6OST), glucosaminyl 3-O sulfotransferase (3OST), and any combination thereof, wherein the one or more heparan sulfate sulfotransferases have a biological activity consisting of producing heparan sulfate upon reacting with PAPS and a heparosan-based polysaccharide, wherein at least one of the heparan sulfate sulfotransferases is fused to affinity tag partner, wherein the affinity tag partner has an equilibrium dissociation constant (K_{D}) with the affinity tag of less than 1 μM; (d) binding the affinity tag partner with the affinity tag, thereby recruiting the one or more heparan sulfate sulfotransferases into the biomimetic condensate; (e) introducing one or more substrates into the aqueous composition, wherein the one or more substrates comprise PAPS and a sulfo group acceptor selected from the group consisting of N-sulfated heparan sulfate (N-HS), N,2-O-sulfated heparan sulfate (N2-HS), N-,2-O,6-O-sulfated heparan sulfate (N26-HS), and any combination thereof; and (t) initiating a chemical reaction between at least one of the heparan sulfate sulfotransferases, PAPS, and at least one of the sulfo group acceptors to synthesize the biological product. In further embodiments, the aqueous composition further comprises a glucuronyl C₅-epimerase enzyme. In various embodiments, the affinity tag is an RIAD peptide motif, and the affinity tag is fused to Homer and Shank. In various embodiments, affinity tag partner is a RIDD peptide motif. In various embodiments, in reactions in which 3OST is selected as a heparan sulfate sulfotransferase, the biological product is heparin.

In another aspect of the invention, the incorporation of catalytic enzymes of interest into an LLPS droplet can be exploited to catalyze the enzymatic synthesis of products that are typically produced using chemical synthesis only. Without being limited by a particular theory, it is believed that enzymes with promiscuous activity can be utilized to react with compounds that are merely in the same general class as their natural substrates. It is believed that this represents the opposite situation of an enzyme like a heparan sulfate sulfotransferase, which exclusively recognizes PAPS as its natural sulfo group donor.

In one non-limiting example, alcohol dehydrogenase (ALD) typically reacts in vivo with ethanol in the presence of nicotinamide adenine dinucleotide (NAD⁺) and a Zn²⁺ cofactor to produce acetaldehyde. However, ALD is widely-known in the art to have activity with several primary and secondary alcohols of varying degrees of steric bulk. Similarly, LipB, particularly the LipB of (Candida antarctica (CALB), typically catalyzes the hydrolysis of fats within biological systems. However, the selectivity and catalytic activity of CALB has been an exceptionally useful tool in the in vitro industrial and laboratory syntheses of esters and amides. In various embodiments, the promiscuous activities of ALD and CALB can be utilized together to catalyze the synthesis of lidocaine, which typically is a two-step elementary-level organic synthesis involving the reaction of 2,6-xylidine with chloroacetic acid to give α-chloro-2,6-dimethylacetanilide, which is subsequently reacted with diethylamine to produce lidocaine. Instead, in various embodiments, ALD can be reacted with 2-(diethylamino)-ethanol and NAD⁺ to produce 2-(diethylamino)-acetic acid, which can in turn react with CALB and 2,6-xylidine to produce lidocaine.

Accordingly, and in various embodiments, a method for catalyzing the in vitro enzymatic synthesis of 2-(diethylamino)-acetic acid within a biomimetic condensate can comprise the following steps: (a) providing an aqueous composition comprising alcohol dehydrogenase (ALD), the ALD having a biological activity consisting of the conversion of a primary or secondary alcohol to an aldehyde or ketone, respectively, wherein the ALD is expressed as a fusion protein comprising the ALD fused to a low-complexity phase separation domain selected from the group consisting of an intrinsically disordered region (IDR) and an elastin-like polypeptide (ELP) domain; (b) assembling the ALD into a biomimetic condensate having an internal portion and an external portion separated by a phase boundary, wherein the ALD is contained within the internal portion, and wherein the internal portion is a liquid; (c) introducing 2-(diethylamino)-ethanol and NAD⁺ into the aqueous composition, wherein the 2-(diethylamino)-ethanol and NAD⁺ can freely traverse the phase boundary between the external portion and internal portion of the biomimetic condensate; and (d) initiating a chemical reaction between ALD, NAD⁺, and 2-(diethylamino)-ethanol to synthesize the 2-(diethylamino)-acetic acid product. In various embodiments, an IDR selected from the group consisting of fused in sarcoma (FUS) protein, TATA-box binding protein associated factor 15 (TAF), P-granule protein LAF-1 (LAF), Ddx4 helicase (DDX), and Tia1 cytotoxic granule-associated RNA binding protein (TIA), particularly FUS, is fused to the ALD enzyme. In various embodiments, the ALD comprises at least a fragment of an amino acid sequence selected from the group consisting of SEQ ID NO: 1, SEQ ID NO: 2, SEQ ID NO: 3, SEQ ID NO: 4, SEQ ID NO: 5, and SEQ ID NO: 6. In various embodiments, the biomimetic condensate further comprises Candida antarctica lipase B (CALB), the one or more substrates further comprises 2,6-xylidine, and the biological product is lidocaine. In a further embodiment, the CALB comprises at least a fragment of the amino acid sequence, SEQ ID NO: 7.

Additionally, and in various embodiments, a method for the in vitro enzymatic synthesis of 2-(diethylamino)-acetic acid within a biomimetic condensate can comprise the steps of: (a) providing an aqueous composition comprising one or more scaffold proteins, the one or more scaffold proteins comprising at least a fragment of a GKAP protein, at least a fragment of a Homer protein, and at least a fragment of a Shank protein, wherein at least one of GKAP, Homer, Shank proteins is fused to an affinity tag; (b) assembling the one or more scaffold proteins into a biomimetic condensate having an internal portion and an external portion separated by a phase boundary, wherein the one or more scaffold proteins are contained within the internal portion, and wherein the internal portion is a liquid; (c) introducing alcohol dehydrogenase (ALD) into the aqueous composition, wherein the ALD has a biological activity consisting of the conversion of a primary or secondary alcohol to an aldehyde or ketone, respectively, wherein the ALD is fused to affinity tag partner, wherein the affinity tag partner has an equilibrium dissociation constant (K_{D}) with the affinity tag of less than 1 μM; (d) binding the affinity tag partner with the affinity tag, thereby recruiting the ALD into the biomimetic condensate; (e) introducing one or more substrates into the aqueous composition, wherein the one or more substrates comprise NAD⁺, and 2-(diethylamino)-ethanol; and (f) initiating a chemical reaction between ALD, NAD⁺, and 2-(diethylamino)-ethanol to synthesize the 2-(diethylamino)-acetic acid product. In various embodiments, the affinity tag is an RIAD peptide motif, and the affinity tag is fused to Homer and Shank. In various embodiments, affinity tag partner is a RIDD peptide motif. In various embodiments, the ALD comprises at least a fragment of the amino acid sequence selected from the group consisting of SEQ ID NO: 1, SEQ ID NO: 2, SEQ ID NO: 3, SEQ ID NO: 4, SEQ ID NO: 5, and SEQ ID NO: 6. In various embodiments, the biomimetic condensate further comprises Candida antarctica lipase B (CALB), the one or more substrates further comprises 2,6-xylidine, and the biological product is lidocaine. In a further embodiment, the CALB comprises at least a fragment of the amino acid sequence, SEQ ID NO: 7 and is also fused to a RIDD peptide motif as an affinity tag partner.

These and other embodiments of the present invention will be apparent to one of ordinary skill in the art from the following detailed description.

### Definitions

The term, “active site,” refers to sites in catalytic proteins, in which catalysis occurs, and can include one or more substrate binding sites. Active sites are of significant utility in the identification of compounds that specifically interact with, and modulate the activity of, a particular polypeptide. The association of natural ligands or substrates with the active sites of their corresponding receptors or enzymes is the basis of many biological mechanisms of action. Similarly, many compounds exert their biological effects through association with the active sites of receptors and enzymes. Such associations may occur with all or any parts of the active site.

The term, “amino acid,” refers to a molecule having the structure wherein a central carbon atom (the alpha-carbon atom) is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group, R. When incorporated into a peptide, polypeptide, or protein, an amino acid loses one or more atoms of its amino and carboxylic groups in the dehydration reaction that links one amino acid to another. As a result, when incorporated into a protein, an amino acid is referred to as an “amino acid residue.” In the case of naturally occurring proteins, an amino acid residue's R group differentiates the 20 amino acids from which proteins are synthesized, although one or more amino acid residues in a protein may be derivatized or modified following incorporation into protein in biological systems (e.g., by glycosylation and/or by the formation of cysteine through the oxidation of the thiol side chains of two non-adjacent cysteine amino acid residues, resulting in a disulfide covalent bond that frequently plays an important role in stabilizing the folded conformation of a protein, etc.). Additionally, when an alpha-carbon atom has four different groups (as is the case with the 20 amino acids used by biological systems to synthesize proteins, except for glycine, which has two hydrogen atoms bonded to the carbon atom), two different enantiomeric forms of each amino acid exist, designated D and L. In mammals, only L-amino acids are incorporated into naturally occurring polypeptides. Enzymes utilized in accordance with methods of the present invention can incorporate one or more D- and L-amino acids, or can be comprised solely of D- or L-amino acid residues.

Similarly, non-naturally occurring amino acids can also be incorporated into any of the enzymes utilized in accordance with the methods of the present invention. Examples of such amino acids include, without limitation, alpha-amino isobutyric acid, 4-amino butyric acid, L-amino butyric acid, 6-amino hexanoic acid, 2-amino isobutyric acid, 3-amino propionic acid, ornithine, norleucine, norvaline, hydroxyproline, sarcosine, citrulline, cysteic acid, t-butyl glycine, t-butyl alanine, phenylglycine, cyclohexyl alanine, beta-alanine, fluoro-amino acids, designer amino acids (e.g., beta-methyl amino acids, alpha-methyl amino acids, alpha-methyl amino acids) and amino acid analogs in general.

The term, “and/or,” when used in the context of a listing of entities, refers to the entities being present singly or in combination. Thus, for example, the phrase “A, B, C, and/or D” includes A, B, C, and D individually, as well as all combinations and sub-combinations of A, B, C, and I).

The terms, “biological activity” or “catalytic activity,” refer to the ability of an enzyme to catalyze a particular chemical reaction by specific recognition of a particular substrate or substrates to generate a particular product or products.

The term, “coding sequence,” refers to that portion of a nucleic acid, for example, a gene, that encodes an amino acid sequence of a protein.

The term, “codon-optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, it is well known that codon usage in vivo is non-random and biased toward one or more codon triplets. In some embodiments of the invention, the polynucleotide encoding for an engineered enzyme may be codon optimized for optimal production from the host organism selected for expression.

The terms, “corresponding to,” “in reference to,” or “relative to,” when used in the context of the numbering of a given amino acid or polynucleotide sequence, refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence.

The term, “deletion,” refers to modification of a polypeptide by removal of one or more amino acids from the reference polypeptide. Deletions can comprise removal of I or more amino acids, the net result of which is retaining the catalytic activity of the reference polypeptide. Deletions can be directed to the internal portions and/or terminal portions of a polypeptide. Additionally, deletions can comprise continuous segments or they can be discontinuous.

The terms, “fragment” or “segment,” refer to a polypeptide that has an amino- or carboxy-terminal deletion, but where the remaining amino acid sequence is identical to the corresponding positions in a reference-sequence. Fragments can be at least 10, 20, 30, 40,50, or more amino acids, and up to 70%, 80%, 90%, 95%, 98%, and 99% of a particular enzyme.

The terms, “functional site” or “functional domain,” generally refer to any site in a protein that confers a function on the protein. Representative examples include active sites (i.e., those sites in catalytic proteins where catalysis occurs) and ligand binding sites. Ligand binding sites include, but are not limited to, metal binding sites, co-factor binding sites, antigen binding sites, substrate channels and tunnels, and substrate binding domains. In an enzyme, a ligand binding site that is a substrate binding domain may also be an active site. Functional sites may also be composites of multiple functional sites, wherein the absence of one or more sites comprising the composite results in a loss of function.

The terms, “gene,” “gene sequence,” and “gene segment,” refer to a functional unit of nucleic acid unit encoding for a functional protein, polypeptide, or peptide. As would be understood by those skilled in the art, this functional term includes both genomic sequences and c)NA sequences. The terms, “gene,” “gene sequence,” and “gene segment,” additionally refer to any DNA sequence that is substantially identical to a polynucleotide sequence disclosed herein encoding for an enzyme gene product, protein, or polysaccharide, and can comprise any combination of associated control sequence. The terms also refer to RNA, or antisense sequences, complementary to such DNA sequences. As used herein, the term “DNA segment” includes isolated DNA molecules that have been isolated free of recombinant vectors, including but not limited to plasmids, cosmids, phages, and viruses.

The term, “insertion,” refers to modifications to the polypeptide by addition of one or more amino acids to the reference polypeptide. Insertions can be in the internal portions of the polypeptide, or to the C- or N-termini of the polypeptide. Insertions can include fusion proteins as is known in the art and described below. The insertions can comprise a continuous segment of amino acids or multiple insertions separated by one or more of the amino acids in the reference polypeptide.

The term, “isolated nucleic acid” as used herein with respect to nucleic acids derived from naturally-occurring sequences, means a ribonucleic or deoxyribonucleic acid which comprises a naturally-occurring nucleotide sequence and which can be manipulated by standard recombinant DNA techniques, but which is not covalently joined to the nucleotide sequences that are immediately contiguous on its 5′ and 3′ ends in the naturally-occurring genome of the organism from which it is derived. As used herein with respect to synthetic nucleic acids, the term “isolated nucleic acid” means a ribonucleic or deoxyribonucleic acid which comprises a nucleotide sequence which does not occur in nature and which can be manipulated by standard recombinant DNA techniques. An isolated nucleic acid can be manipulated by standard recombinant DNA techniques when it may be used in, for example, amplification by polymerase chain reaction (PCR), in vitro translation, ligation to other nucleic acids (e.g., cloning or expression vectors), restriction from other nucleic acids (e.g., cloning or expression vectors), transformation of cells, hybridization screening assays, or the like.

The terms, “naturally occurring” or “natural,” refer to forms of an enzyme found in nature. For example, a naturally occurring or natural polypeptide or polynucleotide sequence is a sequence present in an organism that can be isolated from a source in nature and which has not been intentionally modified by human manipulation. A natural polypeptide or polynucleotide sequence can also refer to recombinant proteins or nucleic acids that can be synthesized, amplified, and/or expressed in vitro, and which have the same sequence and biological activity as an enzyme produced in vivo.

The term, “oligosaccharide” refers to saccharide polymers containing a small number, typically three to nine, sugar residues within each molecule.

The term, “percent identity,” refers to a quantitative measurement of the similarity between two or more nucleic acid or amino acid sequences. As a non-limiting example, the percent identity can be assessed between two or more engineered enzymes, two or more naturally occurring enzymes, or between one or more engineered enzymes and one or more naturally occurring enzymes. Percent identity can be assessed relative to two or more full-length sequences, two or more truncated sequences, or a combination of full-length sequences and truncated sequences.

The term, “polysaccharide,” refers to polymeric carbohydrate structures formed of repeating units, typically monosaccharide or disaccharide units, joined together by glycosidic bonds, and which can range in structure from a linear chain to a highly-branched three-dimensional structure. Although the term “polysaccharide,” as used in the art, can refer to saccharide polymers having more than ten sugar residues per molecule, “polysaccharide” is used within this application to describe saccharide polymers having more than one sugar residue, including saccharide polymers that have three to nine sugar residues that may be defined in the art as an “oligosaccharide.”

The terms, “protein,” “gene product,” “polypeptide,” and “peptide” can be used interchangeably to describe a biomolecule consisting of one or more chains of amino acid residues. In addition, proteins comprising multiple polypeptide subunits (e.g., dimers, trimers or tetramers), as well as other non-proteinaceous catalytic molecules will also be understood to be included within the meaning of “protein” as used herein. Similarly, “protein fragments,” i.e., stretches of amino acid residues that comprise fewer than all of the amino acid residues of a protein, are also within the scope of the invention and may be referred to herein as “proteins.” Additionally, “protein domains” are also included within the term “protein.” A “protein domain” represents a portion of a protein comprised of its own semi-independent folded region having its own characteristic spherical geometry with hydrophobic core and polar exterior.

The term, “recombinant,” when used with reference to, for example, a cell, nucleic acid, or polypeptide, refers to a material that has been modified in a manner that would not otherwise exist in nature. Non-limiting examples include, among others, recombinant cells expressing genes that are not found within the native (non-recombinant) form of the cell or express native genes that are otherwise expressed at a different level.

The term, “reference sequence,” refers to a disclosed or defined sequence used as a basis for sequence comparison. A reference sequence may be a subset of a larger sequence, for example, a segment of a full-length gene or polypeptide sequence. Generally, a reference sequence refers to at least a portion of a full-length sequence, typically at least 20 amino acids, or the full-length sequence of the nucleic acid or polypeptide.

The term, “saccharide,” refers to a carbohydrate, also known as a sugar, which is a broad term for a chemical compound comprised of carbon, hydrogen, and oxygen, wherein the number of hydrogen atoms is essentially twice that of the number of oxygen atoms. Often, the number of repeating units may vary in a saccharide. Thus, disaccharides, oligosaccharides, and polysaccharides are all examples of chains composed of saccharide units that are recognized by heparan sulfate sulfotransferase enzymes as sulfo group acceptors.

The term, “transformation,” refers to any method of introducing an exogenous nucleic acid into a cell including, but not limited to, transformation, transfection, electroporation, microinjection, direct injection of naked nucleic acid, particle-mediated delivery, viral-mediated transduction or any other means of delivering a nucleic acid into a host cell which results in transient or stable expression of said nucleic acid or integration of said nucleic acid into the genome of said host cell or descendant thereof.

### DETAILED DESCRIPTION OF THE INVENTION

The present disclosure describes methods, systems, and compositions utilized in the manufacture of pharmaceutical products within biomimetic, synthetic condensates, including such products synthesized therefrom. The embodiments disclosed herein represent an improvement over classical in vitro biocatalysis, which is dependent on the mixing of enzymes, reagents, and cofactors in a comparatively massive solution. The product formation rate is dictated by the concentrations of the components and dependent on stochastic, random collisions between the molecules. At commercial scale, bulk diffusion and mixing of the components control the rate more than any other factor. Without being limited by a particular theory, it is believed that the rates of product formation exhibited by the biomimetic synthetic condensates of the present invention will be at least tens or hundreds of times greater than traditional enzymatic biocatalysis.

The underlying science and engineering principles of natural compartments within eukaryotic cells are well established, including the membrane-bound organelles endoplasmic reticulum, Golgi apparatus, lysosomes, and mitochondria, and condensates. A vast majority of the attention focuses on the role of condensates in various diseases, particularly dementia. However, and without being limited by a particular theory, it is believed that the design and engineering of synthetic condensates as a manufacturing scaffold represents a new approach in the field.

By way of background, a critical feature of any membrane-enclosed catalytic compartment is that the enzymes are typically concentrated on the membrane surface facing the lumen, which is very narrow (often <50 nm) and typically contains the substrates for the enzymes. As a non-limiting example, within one of the processing compartments (cisterna) of the Golgi, the enzymes in the wall can comprise >90% of the protein mass and greatly exceed the substrates (such as heparin) in the narrow lumen. This greatly favors efficient synthesis and biosynthesis of products like heparin in this natural, highly condensed environment.

Dozens of natural membraneless organelles have been described. As the name suggests, the enzyme and substrate content of these organelles spontaneously separate from the rest of the cell by virtue of the intrinsic physical chemistry of the enzyme's sequence into liquid-like droplets. Because these liquid-liquid phase separated (LLPS) droplets are not surrounded by a bilayer membrane, substrates can enter and products can leave, but the enzymes involved remain in a condensed, highly concentrated, and functional liquid state. Further, the internal structure of coacervates, such as LIPS protein droplets, contains a vast number of branching and anastomosing aqueous channels which are on the low nanoscale in size. These channels am themselves liquid in nature-constantly moving, breaking, rejoining-thereby promoting the rapid mixing of enzymes and substrates, closely mimicking the narrow lumen of efficient processing compartments like Golgi cisternae. As a result, it is believed that substrates are also passively concentrated during phase separation, and that both enzyme and substrate concentrations would be much higher in the condensed state than if they were allowed to freely disperse within the cytoplasm.

Accordingly, and in some embodiments, the biomimetic condensates formed in conjunction with the methods and systems of the present invention are heterogeneous structures formed in vitro as a phase-separated component suspended within a carrier composition, typically an aqueous solution. The condensate can comprise one or more small molecules and/or macromolecules that in some embodiments, can mimic or approximate function of organelles and other bodies within eukaryotic cells, such as, in non-limiting examples, the endoplasmic reticulum (ER), Golgi apparatus and the nucleolus. Because the physiochemical basis for condensate formation is generally governed by the same or similar sequences, structures, and principles as natural biological condensates, the biomimetic condensates described herein can assemble in vitro within the carrier composition by clustering, thereby increasing the local concentration of their constituent components, most commonly by liquid-liquid or solid-liquid phase separation from their external environment.

Consequently, a biomimetic condensate can be formed within a composition to localize into a confined space one or more materials capable of catalyzing the formation of a biochemical product. In some embodiments, the biochemical product is a pharmaceutical compound suitable for administration to a human or animal subject. In some embodiments, the biochemical product is a small molecule, produced by, as non-limiting examples, epimerization, stereoselective addition, oxidation, reduction, acylation, diacylation, amidation, deamidation, sulfation, phosphorylation, boronation, halogenation, dehalogenation, condensation, functional group transfer, hydrolysis, and/or any combination thereof. In some embodiments, the biochemical product is a functional or structural biopolymer. Non-limiting examples of biopolymers producible within a biomimetic condensate of the present invention are messenger RNA, oligonucleotides, polypeptides, polyesters, antibodies, polysaccharides, and/or any combination thereof. In some embodiments, the biochemical product is a chemical intermediate produced in a multi-step synthesis of a desired final biochemical product, including but not limited to any of the classes of biochemical products listed above.

The enabling principle that concentrating enzymes and substrates in a biocondensate will increase product formation rate follows from known mathematical relationships in enzyme kinetics. For instance, at a given Kₘ, increasing substrate or the enzyme concentrations will increase product formation up to the rate limiting step of the reaction. Accordingly, sequestering enzymes and substrates within LLPS droplets results in an accelerated reaction rate.

Conducting chemical reactions within synthetic biocondensates demonstrates an advantage over other enzyme-substrate sequestering approaches, such as lipid bilayers, because such systems require active mechanisms to introduce substrate into the system. As a result, previous attempts to compartmentalize enzyme reactions in synthetic lipid-based membrane-enclosed organelles have been unsuccessful for several reasons: the inability of substrates to enter and products leave the vesicles; the structure of the vesicle took up valuable internal reaction space; the lack of a suitable mechanism to passively increase substrate concentrations in the vesicle; and finally, heterogeneous, multicomponent systems are difficult to characterize and manufacture at scale.

On the other hand, the intrinsic nature of enzyme-containing membraneless vesicles concentrates substrate, promotes active site binding and dynamic cycling, formation and disaggregation of the condensate itself, and product release. Without being limited by a particular theory, it is believed that reaction rate can be optimized further upon engineering the enzyme concentration to be greater than the concentration of substrate within the condensate and/or introducing additional substrate or cofactor binding capabilities (see e.g., Poudyal, R. R. et al. Template-directed RNA polymerization and enhanced ribozyme catalysis inside membraneless compartments formed by coacervates. Nat. Commun, 10, (2019)).

As one may expect, among the voluminous range of biocondensates that have been characterized to date, there have been several mechanisms identified for triggering the self-assembly of proteins into a membraneless organelle, LLPS droplet, or other condensate structure. One non-limiting example of such a mechanism is the presence of an intrinsically disordered low-complexity (LC) amino acid sequence motif can be engineered into the amino acid sequence of the enzyme, typically at or approximate to the C-terminus. LC-amino acid sequence motifs can include, but not limited to, intrinsically-disordered regions and elastin-like polypeptides.

Particularly, intrinsically-disordered regions(IDRs) are low-complexity, non-repeating sequences involving a small number (30-100) of a mixture of charged, aromatic, and polar amino acids that, on their own, have an undefined secondary and tertiary structure. In contrast, a major determinant of polypeptide segments folding co-operatively into a defined tertiary structure is the long-range hydrophobic interaction between amino acids in the linear sequence. Because of their lack of a unique three-dimensional structure either entirely or in parts in their native state, IDRs generally sample a variety of conformations that are in dynamic equilibrium under physiological conditions. In the fifteen years since natural intracellular condensates have been identified, a vast number of IDRs have been identified within the human genome. Indeed, as many as 30% of the coding regions have IDRs.

Yet, while IDRs are indeed dynamic and adopt an array of conformations, the number of structures they can possess are not infinite. Computational analysis of sequences, single-molecule studies, and molecular dynamics simulations has revealed that the amino acid composition affects the IDR conformational states and can determine whether they adopt a totally extended conformation (segments with high net charge and low hydrophobicity) or a compact conformation (depending on the balance between hydrophobicity and net charge). For the same number of charged residues, the charge patterning has also been shown to determine whether the polypeptide segment will be fully extended (e.g. alternating positively and negatively charged residues) or a collapsed globule (e.g. clearly separated stretches of positively and negatively charged residues), or somewhere in between.

In the last few years, it has been demonstrated that many low-complexity regions and IDRs with repeating peptide motifs can form nonmembrane-bound organelles and higher-order assemblies, often in a highly reversible manner. For instance, Q/N-rich regions are important for forming cellular assemblies, such as P-bodies, FG-rich regions are critical in forming the hydrogel-like structure of the nuclear pore, and repeats of multiple linear motifs can mediate phase separation and organize matter in cells, as seen in certain actin regulatory proteins. Thus, DRs can mediate functions comparable to structured domains, such as (i) the formation of protein complexes and higher-order assemblies of variable stoichiometry of subunits (ii) conformational transition (disorder-to-order and order-to-disorder) in response to specific environmental changes, context, or ligands, and (iii) allosteric communication. As a result, and without being limited by a particular theory, it is believed that the methods of the present invention exploit IDRs natural ability to synergistically increase the functional versatility of proteins.

Typically, IDRs responsible for self-assembly of membraneless organelles are modular. In nature, they are accompanied by one or more folded domains that can be enzymes or ligand binding proteins, and it is this feature that enables many different membraneless organelles, each carrying out unique activities, to self-assemble by LLPS from a common cytoplasm. This modular organization separates the property of phase separation (contained in the IDR) from the property of biological specificity (contained in the folded domains). Without being limited by a particular theory, it is believed that these features provide a robust springboard for engineering, as it is widely-known that virtually any IDR can be attached to virtually any folded protein and will lead to enclosing the designated protein in a droplet.

Further, natural IDRs can be engineered to possess tunable properties, including but not limited to the size, viscosity, and porosity of the resulting condensate, as well as the phase separation diagram demonstrating the condition(s) in which the IDR-comprising enzymes will reversibly self-assemble. Changes in pH, temperature, and/or light have all been demonstrated to facilitate condensate formation and product release. In fact, several non-limiting examples of engineered IDRs that can be fused to enzymes and facilitate the formation stable micron-size droplets have already been demonstrated (see, e.g., Simon, J. R., Carroll, N. J., Rubinstein, M., Chilkoti, A. & López, G. P. Programming molecular self-assembly of intrinsically disordered proteins containing sequences of low complexity. Nat. Chem. 9, (2017)).

Accordingly, and in various embodiments, the modular nature of IDRs can be harnessed to create synthetic liquid condensates containing enzymes useful for manufacturing molecules of interest. It is believed that the enzymes within the synthetic condensates approximate the kinetics and thermodynamics of intracellular membraneless vesicles, affording higher specific activity than those observed for enzymes in traditional in vitro enzymatic biocatalytic processes, which are limited by bulk diffusion within a relatively large volume. Various non-limiting examples describing the fusion of IDRs to catalytic enzymes used to synthesize a biological product of interest are described in the Examples section, below.

As described above, another LC-amino acid sequence motif that can be incorporated into the amino acid sequence of a catalytic enzyme is an elastin-like polypeptide (ELP). ELPs and derivatives thereof have been extensively described, for example, in U.S. Pat. Nos. 6,852,834 and 10,526,396, the disclosures of which are incorporated by reference in their entireties. ELPs are composed of a repetitive Val-Pro-Gly-Xaa-Gly sequence, where Xaa is any amino acid except proline, and generally possess lower-critical-solution-temperature (LCST) phase behavior, wherein at a fixed enzyme concentration, increasing the temperature above an cloud-point temperature (also, “inverse transition temperature,” Tₜ) triggers phase separation. The LCST phase behavior of ELPs is a hydrophobicity-driven effect, such that increasing the hydrophobicity of Xaa enhances phase separation by lowering the cloud-point temperature. For example, the cloud-point temperature of [VPGVG]_{N} is lower than that of [VPGSG]_{N} because Ser is less hydrophobic than Val. By using ELPs with different hydrophobicity, a multiphase condensate can be constructed. Various non-limiting examples describing the fusion of ELPs to catalytic enzymes used to synthesize a biological product of interest are described in the Examples section, below.

In addition to condensates formed upon engineering a low-complexity amino acid sequence motif into a catalytic enzyme directly, biomimetic condensates can also be formed in which the condensate is assembled via proteins that do not take part in the synthesis of a desired biological product, but instead provide a scaffold for recruiting into the condensate enzymes that do catalyze biological synthesis. A non-limiting example of such a system is demonstrated by proteins present within the post-synaptic densities (PSD) of neuronal synapses, which possess structures comprising densely packed proteins forming disc-shaped mega-assemblies that are hundreds of nm in width, and which are formed by self-assembling mechanisms but are not enclosed by membrane bilayers. In vitro, several purified PSI) proteins mixed at physiological concentrations can form highly condensed, self-organized PSD-like assemblies via liquid-liquid phase separation (LLPS).

Four of the most abundant proteins within the PSD are PSD-95, GKAP, Shank, and Homer, collectively serving to connect ion-channels/receptors on the postsynaptic plasma membrane with the actin cytoskeleton in the cytoplasm of PSDs. Full-length PSD-95 and Homer, mixed at a 1:1:1:1 with GKAP and Shank constructs fragmented to promote soluble and stable protein, has been observed to readily demonstrate phase separation and form LLPS droplets, with each droplet comprising highly-enriched concentrations of all four proteins and the number of droplets directly proportional to the concentration of each protein (see Zeng, M., et al., (2018) Cell 174:1172-1187, the disclosure of which is incorporated by reference in its entirety). LLPS droplet formation was also demonstrated with only GKAP, Shank, and Homer, having the amino acid sequences of SEQ ID NO: 28, SEQ ID NO: 29, and SEQ ID NO: 31.

Additionally, GKAP/Shank/Homer condensates can be utilized to recruit other proteins into the condensate using an affinity tag-affinity tag partner system. Notably, an affinity tag can be fused to GKAP, Shank, and Homer without affecting their ability to form LLPS droplets, and selection of an appropriate affinity tag partner to fuse to a target protein can recruit the target protein to the LIPS droplet. As a non-limiting example, the high-affinity peptide-peptide interacting pair, RIDD and RIAD, can be utilized, in which RIAD is the affinity tag fused to one or more of GKAP, Shank, and Homer, and RIDD is the affinity tag partner fused to the target protein. RIDD and RIAD are two peptide motifs derived from the natural proteins, cAMP-dependent protein kinase and the A kinase-anchoring proteins, respectively. The 50-residue long RIDD motif (residual AAs 12-61 of Ria, SEQ ID NO: 33) forms a stable dimer, and the dimer binds with the 18-residue long RIAD (SEQ ID NO: 32) with an equilibrium dissociation constant, K_{d} of 1.0 nM. Recruitment of a target protein into a GKAP/Shank/Homer condensate using RIDD and RIAD has been demonstrated when the target protein is enhanced green fluorescent protein (EGFP), which enabled the visualization of the enhancement of EGFP-RIDD concentration within GKAP/Homer-RIAD/Shank-RIA) LLPS droplets (See, e.g., Liu. M., et al., (2020) Biomacromolecules 21:2391-2399, the disclosure of which is incorporated by reference in its entirety). Moreover, the same authors demonstrated that multiple target enzymes (MenF, MenD, and MenH) could all be recruited into the same LLPS droplets, even though only MenH was tagged with RIDD. Accordingly, and in various embodiments, the GKAP/Shank/Homer LIPS droplets can be utilized as a vehicle for enhancing the concentration of catalytic enzymes inside a condensate and catalyzing a more efficient synthesis of a desired biological product. Various non-limiting examples describing the utilization of affinity tag-affinity tag partners to drive catalysis within GKAP/Shank/Homer LLPS droplets are further described in the Examples section, below.

### EXAMPLES

The following working and prophetic examples illustrate the embodiments of the invention that are presently best known. However, it is to be understood that the following examples are non-limiting, and merely exemplary or illustrative of the application of the principles of the present invention. Numerous modifications and alternative compositions, methods, and systems may be devised by those skilled in the art without departing from the spirit and scope of the present invention. Additionally, to the extent that section headings are used, they should not be construed as necessarily limiting. Any use of the past tense to describe an example otherwise indicated as constructive or prophetic is not intended to reflect that the constructive or prophetic example has actually been carried out.

Thus, while the present invention has been described above with particularity, the following examples provide further detail in connection with what are presently deemed to be the most practical and preferred embodiments of the invention.

### Example 1: Expression and Purification of Enzymes Capable of Assembling within a Pre-Formed Biomimetic Condensate and Synthesizing Heparan Sulfate

A study is conducted in accordance with embodiments of the present disclosure to obtain purified enzymes capable of assembling within a biomimetic condensate and synthesizing heparan sulfate. The cloning, expression, and purification of heparan sulfate sulfotransferases with sulfo donor preference for either 3′-phosphoadenosine 5′-phosphosulfate (PAPS), adenylyl sulfate, or an aryl sulfate compound has been extensively described, including, as non-limiting examples, within U.S. Pat. Nos. 5,541,095, 5,817,487, 5,834,282, 6,861,254, 8,771,995, 9,951,149, 11,473,068, 11,542,534, 11,572,549, 11,572,550, 11,629,364, 11,692,180, 11,708,567, 11,708,593, 11,767,518, and 11,773,382, U.S. Pat Pub Nos. 2009/0035787, 2013/0296540, and 2016/0122446, as well as unpublished U.S. Provisional Patent No. 63/641,453, the disclosures of which are all incorporated by reference in their entireties.

Amino acid sequences for three heparan sulfate sulfotransferases, 2OST, 6OST, and 3OST, as well as one glucuronyl C₅-epimerase (hereinafter, “Epi”), are engineered with a 50-residue RIDD peptide motif, corresponding to SEQ ID NO: 33, fused at either the N- or C-terminus. Methods for cloning and expressing a fusion protein generally are well known in the art, and several examples of fusions comprising the RIDD peptide motif fused to an enzyme of interest are particularly described in Liu, M., et al., (2020) Biomacromolecules 21:2391-2399, the disclosure of which is incorporated by reference in its entirety. In Liu, fusion expression constructs were formed by ligating a nucleotide sequence encoding for the RIDD peptide motif into a pET32-based plasmid comprising a gene encoding for the enzyme of interest, as well as sequences encoding for a poly-histidine tag to facilitate purification in a Ni²⁺ affinity column. Constructed plasmids were expressed as soluble protein in LB media within E. coli BL21 (DE3) cells.

Plasmids comprising nucleotide sequences encoding for a fusion protein comprising the RIDD peptide motif and one of 2OST, 6OST, 3OST, or Epi are constructed, expressed, and purified using standard biochemical techniques. Any 2OST, 6OST, 3OST, or Epi amino acid sequence can be fused to a RIDD peptide motif. Non-limiting examples of such 2OST enzymes that can be fused to RIDD are SEQ ID NO: 8, SEQ 11) NO: 9, and SEQ ID NO: 10. Non-limiting examples of such 6OST enzymes that can be fused to RIDD are SEQ ID NO: 11, SEQ ID NO: 12, and SEQ ID NO: 13. Non-limiting examples of such 3OST enzymes that can be fused to RIDD are SEQ ID NO: 14, SEQ ID NO: 15, and SEQ HD NO: 16. Non-limiting examples of such Epi enzymes that can be fused to RIDD are SEQ ID NO: 17. It is expected that soluble RIDD-2OST, RIDD-6OST, RIDD-3OST, and RIDD-Epi are all obtained in suitable quantities to both assemble into biomimetic condensates and synthesize heparan sulfate products.

### Example 2: Expression and Purification of Enzymes Capable of Assembling within a Pre-Formed Biomimetic Condensate and Forming Lidocaine

A study is conducted in accordance with embodiments of the present disclosure to obtain purified enzymes capable of assembling within a biomimetic condensate and synthesizing lidocaine. Amino acid sequences for two enzymes, alcohol dehydrogenase (hereinafter, “ALD”) and Candida antarctica lipase B (hereinafter, “CALB” or interchangeably, “LipB”), are engineered with a RIDD peptide motif corresponding to SEQ ID NO: 33 fused at either the N- or C-terminus. Plasmids comprising nucleotide sequences encoding for a fusion protein comprising the RIDD peptide motif and one of ALD or CALB are constructed, expressed, and purified using standard biochemical techniques. Any ALD amino acid sequence can be fused to a RIDD peptide motif. Non-limiting examples of such ALD enzymes that can be fused to RIDD are SEQ ID NO: 1, SEQ ID NO: 2, SEQ ID NO: 3, SEQ ID NO: 4, SEQ ID NO: 5, and SEQ ID NO: 6. The amino acid sequence for CALB is SEQ ID NO: 7. In some embodiments, however, the amino acid sequence for CALB can be substituted by the amino acid sequence of a LipB from any other organism. It is expected that soluble RIDD-ALD and RIDD-CALB are all obtained in suitable quantities to both assemble into biomimetic condensates and synthesize lidocaine as a product.

### Example 3: Assembly of a GKAP-Homer3-Shank Scaffold into a Biomimetic Condensate

A study is conducted in accordance with embodiments of the present disclosure to form a scaffold comprising GKAP, Homer3, and Shank proteins and induce their assembly into liquid-liquid phase separated (LLPS) droplets. GKAP, Homer3, and Shank are each cloned, expressed, and purified according to the procedure described by Liu, et al., above.

The amino acid sequences for Homer3 and Shank are engineered to comprise a fusion to an 18-residue peptide, RIAD, having the amino acid sequence of SEQ ID NO: 32. Any Homer3 or Shank amino acid sequence can be fused to a RIAD peptide motif. Further, either the entirety, or one or more fragments, of the amino acid sequences of Homer3 and/or Shank can be expressed. A non-limiting example of one such Homer3 enzyme that can be fused to RIAD is SEQ ID NO: 29, which comprises residues 1-361 of the Homer3 having the UniProt accession number Q9NSC5-1, where residue 342 is mutated from serine to arginine. A non-limiting example of one such Shank enzyme that can be fused to RIAD is SEQ ID NO: 31, which is truncated to include only an N-terminal extended PDZ domain, a Homer-binding sequence, a Cortactin-binding sequence, and a C-terminal sterile alpha motif (SAM) domain. To increase solubility in aqueous composition, Shank can also be appended to the 56-residue BI domain of Streptococcal Protein G (GB1).

GKAP can be cloned and expressed without the RIAD peptide motif, using any full-length or truncated GKAP amino acid sequence. A non-limiting example of one such GKAP enzyme is SEQ ID NO: 28, which comprises the sequences of the PSD-95 enzyme guanylate kinase (GK)-binding repeats and a C-terminal extended PDZ-binding motif.

To, prepare the enzymes to assemble as a scaffold within an LIPS droplet, purified samples of GKAP, RIAD-Homer3, and RIAD-Shank are each separately prepared in an aqueous buffer and centrifuged at high speed to remove insoluble aggregates formed during protein expression. Phase separation of the enzymes into an LLPS droplet can be induced simply upon mixing the three enzymes at an equimolar concentration, between 5 and 100 μM (see Zeng, M., et al., (2018) Cell 174:1172-1187, the disclosure of which is incorporated by reference in its entirety), to form an assembly akin to the natural postsynaptic densities (PSD) of neuronal synapses, which comprise GKAP, Homer, and Shank as the most abundant proteins within the network. It is expected that upon combining purified GKAP, RIAD-Homer3, and RIAD-Shank, the three enzymes will coalesce into a plurality of L LPS droplets within the larger aqueous composition.

### Example 4: Recruitment of RIDD-Tagged Enzymes into a GKAP-Homer3-Shank Scaffold

A study is conducted in accordance with embodiments of the present disclosure to incorporate one or more of the RIDD-tagged enzymes of Examples 1 or 2 into the LLPS droplets formed in Example 3. A RIDD-tagged enzyme is added into a solution with pre-formed LPS droplets comprising GKAP, RIAD-Homer3, and RIAD-Shank, where the concentration of the RIDD-tagged enzyme is 1.2×-100× more dilute than the enzymes within the LLPS droplet. As a non-limiting example, if the concentrations of each of GKAP, RIAD-Homer3, and RIAD-Shank within the entire aqueous composition is 5 μM, then the RIDD-tagged enzyme is added to the composition at a concentration anywhere within a range from 0.1 μM to 4 μM. It is expected that the extremely high affinity between the RIDD and RIAD peptide motifs facilitate the recruitment of the RIDD-tagged enzyme into the LLPS droplets without compromising the droplet phase. It is also expected that the local concentration of the RIDD-tagged enzymes within the droplets is enriched 50- to 100-fold relative to the overall concentration within the aqueous composition, in line with observations made by both Liu, et al. and Zeng, et al.

### Example 5: Synthesis of Heparan Sulfate within a GKAP-Homer3-Shank Scaffold

A study is conducted in accordance with embodiments of the present disclosure to synthesize heparan sulfate within the LLPS droplets of Example 3. RIDD-tagged 6OST is recruited into the LLPS droplets, according to the procedure of Example 4. In a non-limiting example, the RIDD-tagged 6OST enzyme is a wild-type 6OST having the amino acid sequence of SEQ ID NO: 11, SEQ ID NO: 12, or SEQ ID NO: 13. Subsequently, PAPS and N-,2-O-sulfated heparan sulfate (N2-HS) are added to the aqueous composition as sulfo group donor and acceptor, respectively, and incubated alongside the LLPS droplets overnight. It is expected that the sulfated product, N-, 2-O,6-O-sulfated heparan sulfate (N26-HS), can be purified from the resulting composition, digested by one or more heparinase enzymes, and confirmed using strong anion exchange chromatography or mass spectrometry.

It is also expected that separate experiments can be conducted in which RIDD-tagged 2OST or RIDD-tagged 3OST is substituted in place of RIDD-tagged 6OST would also generate the appropriate heparan sulfate product. Within reactions in which the sulfotransferase is RIDD-tagged 2OST, the sulfo group acceptor is 2,6-desulfated heparin, which contains epimerized and non-epimerized hexuronic acid residues, and the expected heparan sulfated product is N2-HS. Within reactions in which the sulfotransferase is RIDD-tagged 3OST, the sulfo group acceptor is N26-HS and the expected heparan sulfated product is N-, 2-O, 6-O, 3-O-sulfated heparan sulfate (N263-HS). As with the above example with RIDD-6OST, it is expected that the sulfated product of either RIDD-2OST or RIDD-3OST can be purified from the resulting composition, digested by one or more heparinase enzymes, and confirmed using strong anion exchange chromatography or mass spectrometry.

### Example 6: Multi-Enzyme Synthesis of Heparin within a GKAP-Homer3-Shank Scaffold

A study is conducted in accordance with embodiments of the present disclosure to synthesize heparin within the LLPS droplets of Example 3. RIDD-tagged 2OST, RIDD-tagged Epi, RIDD-tagged 6OST, and RIDD-tagged 3OST are recruited into the LLPS droplets, according to the procedure of Example 4, resulting in condensates comprising GKAP, RIAD-Homer3, RIAD-Shank, RIDD-tagged 2OST, RIDD-tagged Epi, RIDD-tagged 6OST, and RIDD-tagged 3OST. In a non-limiting example, each of the RIDD-tagged 2OST, RIDD-tagged 6OST, and RIDD-tagged 3OST are wild-type sulfotransferases having the amino acid sequences of SEQ ID NO: 8, SEQ ID NO: 9, or SEQ ID NO: 10; SEQ ID NO: 11, SEQ ID NO: 12, or SEQ ID NO: 13; or SEQ ID NO: 14, SEQ ID NO: 15, or SEQ ID NO: 16: respectively. Subsequently, PAPS and N-sulfated heparan sulfate (N-HS) are added to the aqueous composition and incubated alongside the LLPS droplets overnight. It is expected that the heparin product can be purified from the resulting composition, digested by one or more heparinase enzymes, and confirmed using strong anion exchange chromatography or mass spectrometry.

In the above examples, and without being limited by a particular theory, it is believed that the relative proximity of the 2OST, 6OST, and 3OST enzymes readily facilitates the shuttling of the sulfated product of one sulfotransferase to a succeeding sulfotransferase as its starting material. Consequently, the concerted reaction of the 2OST and Epi enzymes to generate N2-HS with sulfated iduronic acid residues provides nearby 6OST enzymes with starting material to generate N26-HS, which is subsequently utilized by the also nearby 3OST enzymes to generate N263-HS. It is also believed that enclosing the three sulfotransferases within the same condensate closely resembles their relative proximity in vivo within the Golgi apparatus, promoting the formation of polysaccharides identical to, or closely mimicking, heparin.

Further, Liu et al., above, demonstrated that multiple enzymes can be recruited to the same LLPS droplets even when one or more of the enzymes are not tagged with RIDD, based on the natural interactions between the enzymes of interest (e.g., three enzymes, MenF, MenD, and MenH were all spontaneously assembled within the same condensates even though the only protein tagged with RIDD was MenH). Similarly, it has been shown that 2OST, Epi, and 6OST can form a complex in vivo, ensuring that as the hexuronic acid residue of N-HS is reversibly epimerized between glucuronic acid and iduronic acid, 2-O sulfation of iduronic acid provides a readily-available substrate for 6OST to synthesize the characteristic IdoA(2S)-GlcNS(6S) disaccharide motif that is a benchmark property of pharmaceutical heparin. Accordingly, and in some embodiments, it is expected that fewer than all four of 2OST, Epi, 6SOT, and 3OST can be tagged with RIDD without impeding their ability to assemble within an LLPS droplet. In one non-limiting example, enzymes tagged with RIDD are 3OST and one or two of 2OST, Epi, and 6OST. In another non-limiting example, at least 3OST and 2OST are tagged with RIDD. In another non-limiting example, at least 3OST and 6OST are tagged with RIDD. In another non-limiting example, at least 3OST and Epi are tagged with RIDD.

### Example 7: Utilization of a Sulfo Donor Regeneration System During the Multi-Enzyme Synthesis of Heparin within a GKAP-Homer3-Shank Scaffold

A study is conducted in accordance with embodiments of the present disclosure to incorporate into the procedure of Example 5 or Example 6 a system for regenerating PAPS. Mechanisms for regenerating PAPS are well-known in the art and have been described, for example, in U.S. Pat. Nos. 6,255,088 and 9,193,958, as well as U.S. Patent Pub. No. 2023/0272444, the disclosures of which are incorporated by reference in their entireties. Particularly, combining an aryl sulfotransferase (AST) enzyme and an aryl sulfate compound, typically pNPS, has been demonstrated to regenerate PAPS from 3′-phosphoadenosine 5′-phosphate (PAP), which is formed as a byproduct of during the in vitro biosynthesis of heparin (see, e.g., U.S. Pat. No. 8,771,995, above).

In one non-limiting example, AST is engineered with a RIDD peptide motif corresponding to SEQ ID NO: 33 fused at either the N- or C-terminus. A plasmid comprising nucleotide sequences encoding for a fusion protein comprising the RIDD peptide motif and AST is constructed, expressed, and purified using standard biochemical techniques. Any AST amino acid sequence can be fused to a RIDD peptide motif. Non-limiting examples of AST enzymes that can be fused to RIDD is SEQ ID NO: 1, SEQ ID NO: 2, SEQ ID NO: 3, SEQ ID NO: 4, SEQ ID NO: 5, or SEQ ID NO: 6. It is expected that soluble RIDD-AST is obtained in suitable quantities to both assemble within biomimetic condensates and regenerate PAPS from PAP.

Subsequently, PAPS, sulfo group acceptor, and pNPS are added to the aqueous composition and incubated alongside the LLPS droplets formed according to the procedure of Example 5 or Example 6. It is expected that the sulfated heparan sulfate or heparin product can be purified from the resulting composition, digested by one or more heparinase enzymes, and confirmed using strong anion exchange chromatography or mass spectrometry. It is also expected that a larger quantity of the heparan sulfate or heparin product is isolated relative to corresponding preparations according to Example 5 or Example 6 without a sulfo donor regeneration system.

### Example 8: Synthesis of 2-(Dimethylamino)-Acetic Acid within a GKAP-Homer3-Shank Scaffold

A study is conducted in accordance with embodiments of the present disclosure to synthesize 2-(diethylamino)-acetic acid within the LLPS droplets of Example 3. RIDD-tagged ALD is recruited into the LLPS droplets according to the procedure of Example 4, resulting in condensates comprising GKAP, RIAD-Homer3, RIAD-Shank, and RIDD-tagged ALD. In a non-limiting example, the RIDD-tagged ALD comprises an ALD having the amino acid sequence of SEQ ID NO: 1, SEQ ID NO: 2, SEQ ID NO: 3, SEQ ID NO: 4, SEQ ID NO: 5, or SEQ ID NO: 6, or a biological functional fragment thereof. Subsequently, 2-(diethylamino)-ethanol and nicotinamide adenine dinucleotide (NAD⁺) are added to the aqueous composition and incubated alongside the LLPS droplets. Without being limited by a particular theory, it is believed that 2-(diethylamino)-acetic acid can be synthesized enzymatically by ALD from 2-(diethylamino)-ethanol because of ALD's well-documented promiscuous activity with other primary alcohols in addition to ethanol (see, e.g., Woan-Jung, G., et al., (1975) Biochemical Pharmacology 24 (3):413-417). Accordingly, it is expected that the 2-(diethylamino)-acetic acid can be purified from the resulting composition and confirmed by one or more analytical techniques, including but not limited to mass spectrometry and nuclear magnetic resonance (NMR) spectrometry.

### Example 9: Multi-Enzyme Synthesis of Lidocaine within a GKAP-Homer3-Shank Scaffold

A study is conducted in accordance with embodiments of the present disclosure to synthesize lidocaine within the LIPS droplets of Example 3. RIDD-tagged ALD and RIDD-tagged CALB are recruited into the LLPS droplets, according to the procedure of Example 4, resulting in condensates comprising GKAP, RIAD-Homer3, RIAD-Shank, RIDD-tagged ALD and RIDD-tagged CALB. In a non-limiting example, each of the ALD and CALB enzymes comprise the amino acid sequences of SEQ ID NO: 1, SEQ ID.) NO: 2, SEQ ID NO: 3, SEQ ID NO: 4, SEQ ID NO: 5, or SEQ ID NO: 6 (ALD) and SEQ ID NO: 7 (CALB). Subsequently, 2-(diethylamino)-ethanol, NAD⁺, and 2,6-xylidine are added to the aqueous composition and incubated alongside the LLPS droplets. Without being limited by a particular theory, it is believed that lidocaine can be synthesized enzymatically by CALB from 2-(diethylamino)-acetic acid (produced according to the procedure of Example 8, above) because of CALB's well-documented promiscuous synthase activity to form a peptide bond upon joining a first molecule having a carboxylic acid functional group with a second molecule having an amine functional group (see, e.g., Orsy, G., et al., (August 2023) Molecules 28 (15):5706). It also expected that the relative proximity of the ALD and CALB enzymes readily facilitates the shuttling of the 2-(diethylamino)-acetic acid product of ALD directly to CALB, where it can be used as a substrate to synthesize lidocaine. Accordingly, it is expected that the lidocaine product of CALB can be purified from the resulting composition and confirmed by one or more analytical techniques, including but not limited to mass spectrometry and nuclear magnetic resonance (NMR) spectrometry.

### Example 10: Engineering an Intrinsically Disordered Region into ALD and CALB

A study is conducted in accordance with embodiments of the present disclosure to obtain purified enzymes having the dual functionality of self-assembling into a biomimetic condensate and to synthesize a biological product, lidocaine, within the condensate itself, Amino acid sequences for ALD and CALB are engineered to further comprise an intrinsically disordered region (IDR), which is a polypeptide segment that typically contains a higher proportion of polar or charged amino acids and which can adopt several possible conformations in response to its chemical environment. Non-limiting examples of such IDRs are fused in sarcoma (FUS) protein, TATA-box binding protein associated factor 15 (TAF), P-granule protein LAF-1 (LAF), Ddx4 helicase (DDX), and Tia1 cytotoxic granule-associated RNA binding protein (TIA), having the amino acid sequences of SEQ ID NO: 18, SEQ ID NO: 20, SEQ ID NO: 22, SEQ ID NO: 24, and SEQ ID NO: 25, respectively. In various embodiments, FUS, TAF, and LAF can be truncated to SEQ ID NO: 19, SEQ ID NO: 21, and SEQ ID NO: 23, respectively Any IDR peptide motif, including but not limited to those enumerated above, can be fused at either the N- or C-terminus of an enzyme of interest.

Plasmids comprising nucleotide sequences encoding for a fusion protein comprising an IDR peptide motif and one of ALD or CALB are constructed, expressed, and purified using standard biochemical techniques. Like the RIDD peptide motifs above in Example 2, an IDR can be fused to any ALD or LipB amino acid sequence. Non-limiting examples of such ALD enzymes that can be fused to an IDR are SEQ ID NO: 1, SEQ ID) NO: 2, SEQ ID NO: 3, SEQ ID NO: 4, SEQ ID NO: 5, or SEQ ID NO: 6 (ALD), while the amino acid sequence for CALB is SEQ ID NO: 7. It is expected that soluble IDR-ALD and IDR-CALB are all obtained in suitable quantities to both assemble into biomimetic condensates and synthesize lidocaine as a product.

### Example 11: Multi-Enzyme Synthesis of Lidocaine within an IDR-Based Condensate

A study is conducted in accordance with embodiments of the present disclosure to synthesize lidocaine within LLPS droplets formed from IDRs fused to an enzyme of interest. In a non-limiting example, the synthesis of the lidocaine intermediate, 2-(diethylamino)-acetic acid, is performed using an ALD having the amino acid sequence of SEQ ID NO: 1, SEQ ID NO: 2, SEQ ID NO: 3, SEQ ID NO: 4, SEQ ID NO: 5, or SEQ ID NO: 6 fused to a FUS IDR having the amino acid sequence of SEQ ID NO: 19 (collectively, “FUS-ALD”). Condensate formation is initiated by adding the FUS-ALD to an aqueous composition at a concentration sufficient to cause self-assembly of FUS IDRs into a condensate, generally above 5 μM in the absence of crowding agents and at physiologically-relevant salt concentrations (see, e.g., Wang, J., et al., (Jul. 26, 2018) Cell 174 (3):688-699). Subsequently, 2-(diethylamino)-ethanol, NAD⁺, and 2,6-xylidine are added to the aqueous composition and incubated alongside the formed LLPS droplets. It is expected that the 2-(diethylamino)-acetic acid can be purified from the resulting composition and confirmed by one or more analytical techniques, including but not limited to mass spectrometry and nuclear magnetic resonance (NMR) spectrometry.

In another non-limiting example, prior to the addition of substrates, CALB, having the amino acid sequence of SEQ ID NO: 7 is fused to either a FUS IDR (SEQ ID NO: 19) or a different IDR such as the LAF IDR (SEQ ID NO: 23) to form a FUS-CALB or LAF-CALB, respectively. Condensation of FUS-CALB or LAF-CALB is initiated upon adding the selected enzyme to an aqueous composition already comprising FUS-ALD droplets, and the composition is incubated for a time sufficient to form combined droplets comprising FUS-ALD and either FUS-CALB or LAF-CALB. Subsequently, 2-(diethylamino)-ethanol, NAD⁺, and 2,6-xylidine are added to the aqueous composition and incubated alongside the LLPS droplets comprising both enzymes. It is expected that the lidocaine product of CALB can be purified from the resulting composition and confirmed by one or more analytical techniques, including but not limited to mass spectrometry and nuclear magnetic resonance (NMR) spectrometry.

### Example 12: Engineering an Intrinsically Disordered Region into 2OST, Epi, 6OST, and 3OST

A study is conducted in accordance with embodiments of the present disclosure to obtain purified enzymes having the dual functionality of self-assembling into a biomimetic condensate and to synthesize the biological products, heparan separate and/or heparin, within the condensate itself. Amino acid sequences for 2OST, Epi, 6OST, and 3OST are engineered to further comprise an IDR. Any IDR peptide motif, including but not limited to the non-limiting examples enumerated above in Example 10, can be fused at either the N- or C-terminus of 2OST, Epi, 6OST, or 3OST.

Plasmids comprising nucleotide sequences encoding for a fusion protein comprising an IDR peptide motif and one of 2OST, Epi, 6OST, or 3OST are constructed, expressed, and purified using standard biochemical techniques. Like the TDR motif in Example 10, above, an IDR can be fused to any 2OST, Epi, 6OST, or 3OST amino acid sequence. Non-limiting examples of such 2OST enzymes that can be fused to an TDR are SEQ ID NO: 8, SEQ ID NO: 9, or SEQ ID NO: 10. Non-limiting examples of such 6OST enzymes that can be fused to an IDR are SEQ ID NO: 11, SEQ ID NO: 12, or SEQ ID NO: 13. Non-limiting examples of such 3OST enzymes that can be fused to an IDR are SEQ ID NO: 14, SEQ ID NO: 15, or SEQ ID NO: 16. Non-limiting examples of such Epi enzymes that can be fused to an IDR are SEQ ID NO: 17. It is expected that soluble IDR-2OST and IDR-6OST, IDR-3OST, and IDR-Epi are all obtained in suitable quantities to both assemble into biomimetic condensates and synthesize heparan sulfate and/or heparin as a product.

### Example 13: Synthesis of Heparan Sulfate within an IDR-Based Condensate

A study is conducted in accordance with embodiments of the present disclosure to synthesize heparan sulfate within LLPS droplets formed from IDRs fused to an enzyme of interest. In a non-limiting example, the synthesis of a heparan sulfate intermediate, N26-HS, formed during the biosynthesis of heparin is performed using a 6OST having the amino acid sequence of SEQ ID NO: 11, SEQ ID NO: 12, or SEQ ID NO: 13 fused to a FUS IDR having the amino acid sequence of SEQ 11) NO: 19 (collectively, “FUS-6OST”). Condensate formation is initiated by adding the FUS-6OST to an aqueous composition at a concentration sufficient to cause self-assembly of FUS IDRs into a condensate, generally above 5 μM. Subsequently, PAPS and N2-HS are added to the aqueous composition and incubated alongside the formed LLPS droplets. It is expected that the N26-HS product can be purified from the resulting composition, digested by one or more heparinase enzymes, and confirmed using strong anion exchange chromatography or mass spectrometry.

In another non-limiting example, separate experiments can be conducted in which a FUS-2OST or FUS-3OST fusion is substituted in place of FUS-6OST to generate the appropriate heparan sulfate product. Non-limiting examples of 2OST enzymes that can be fused to an IDR such as FUS are SEQ ID NO: 8, SEQ ID NO: 9, or SEQ 11) NO: 10, and non-limiting examples of 3OST enzymes that can be fused to IDR such as FUS are SEQ ID NO: 14, SEQ ID NO: 15, or SEQ ID NO: 16. Within reactions in which the sulfotransferase is FUS-2OST, the sulfo group acceptor is 2,6-desulfated heparin, which contains epimerized and non-epimerized hexuronic acid residues, and the expected heparan sulfated product is N2-HS. Within reactions in which the sulfotransferase is FUS-3OST, the sulfo group acceptor is N26-HS and the expected heparan sulfated product is N263-HS. As with the above example with FUS-6OST, it is expected that the sulfated product of either FUS-2OST or FUS-3OST can be purified from the resulting composition, digested by one or more heparinase enzymes, and confirmed using strong anion exchange chromatography or mass spectrometry.

Another experiment is conducted in which IDR-tagged 2OST, 6OST, and 3OST is added to the same aqueous composition to form condensates comprising all three enzymes and within which heparin can be synthesized. In a non-limiting example, the three enzymes are FUS-2OST, FUS-6OST, and FUS-3OST. In another non-limiting example, each of the enzymes is fused to a different IDR, such as for instance, FUS-2OST, LAF-6OST (further comprising the LAF IDR having the amino acid sequence of SEQ ID NO: 23) and TAF-3OST (further comprising the TAF IDR having the amino acid sequence of SEQ ID NO: 21). In either instance, condensation of IDR-tagged 2OST, 6OST, and 3OST is initiated upon adding all three enzymes to an aqueous composition and incubating the composition until mixed LLPS droplets are formed. Heparin synthesis is initiated by adding PAPS and completely desulfated, N-resulfated heparin, which can be chemically prepared from commercial samples of heparin. It is expected that the heparin product can be purified from the resulting composition, digested by one or more heparinase enzymes, and confirmed using strong anion exchange chromatography or mass spectrometry.

### Example 14: Engineering an Elastin-Like Polypeptide into ALD and CALB and Synthesizing Lidocaine

A study is conducted in accordance with embodiments of the present disclosure to obtain purified enzymes having the dual functionality of self-assembling into a biomimetic condensate and to synthesize a biological product, lidocaine, within the condensate itself. Amino acid sequences for ALD and CALB are engineered to further comprise an elastin-like polypeptide (ELP). ELPs are well-known in the art for their use in protein purification and are commercially-available. Generally, ELPs comprise a plurality of repeats of the amino acid sequence Val-Pro-Gly-Xaa-Gly, wherein Xaa can be any amino acid except proline (SEQ ID NO: 26). Any ELP motif, including but not limited to an ELP motif having one or more repeats of the amino acid sequence of SEQ ID NO: 26, can be fused at either the N- or C-terminus of an enzyme of interest.

Plasmids comprising nucleotide sequences encoding for a fusion protein comprising an ELP motif and either of ALD or CALB are constructed, expressed, and purified using standard biochemical techniques. Like RIDD peptide motifs in Example 2 and the IDR motif in Example 10, an ELP motif can be fused to any ALD or LipB amino acid sequence. Non-limiting examples of such ALD enzymes that can be fused to an ELP motif are SEQ ID NO: 1, SEQ ID NO: 2, SEQ ID NO: 3, SEQ ID NO: 4, SEQ ID NO: 5, or SEQ ID NO: 6, while the amino acid sequence for CALB is SEQ ID NO: 7. It is expected that soluble ELP-ALD and ELP-CALB are both obtained in suitable quantities to both assemble into biomimetic condensates and synthesize lidocaine as a product.

Subsequently, the procedure of Example 9 or Example 11 is adapted to synthesize lidocaine. Condensation of ELP-ALD and ELP-CALB is initiated upon adding both enzymes to an aqueous composition and incubating the composition until mixed LLPS droplets are formed. Subsequently, 2-(diethylamino)-ethanol, NAD⁺, and 2,6-xylidine are added to the aqueous composition and incubated alongside the LLPS droplets comprising both enzymes. It is expected that the lidocaine product of CALB can be purified from the resulting composition and confirmed by one or more analytical techniques, including but not limited to mass spectrometry and nuclear magnetic resonance (NMR) spectrometry.

### Example 15: Engineering ELPs into 2OST, 6OST, and 3OST and Synthesizing Heparan Sulfate

A study is conducted in accordance with embodiments of the present disclosure to obtain purified enzymes having the dual functionality of self-assembling into a biomimetic condensate and to synthesize the biological products, heparan separate and/or heparin, within the condensate itself. Amino acid sequences for 2OST, 6OST, and 3OST are each engineered to further comprise an ELP motif. Any ELP motif, including but not limited to an ELP motif having one or more repeats of the amino acid sequence of SEQ ID NO: 26, can be fused at either the N- or C-terminus of 2OST, 6OST, or 3OST.

Plasmids comprising nucleotide sequences encoding for a fusion protein comprising an ELP motif and either of 2OST, 6OST, or 3OST are constructed, expressed, and purified using standard biochemical techniques. An ELP motif can be fused to any 2OST, 6OST, or 3OST amino acid sequence. Non-limiting examples of such 2OST enzymes that can be fused to an ELP motif are SEQ ID NO: 8, SEQ ID NO: 9, or SEQ ID NO: 10. Non-limiting examples of such 6OST enzymes that can be fused to an ELP motif are SEQ ID NO: 11, SEQ ID NO: 12, or SEQ ID NO: 13. Non-limiting examples of such 3OST enzymes that can be fused to an ELP motif are SEQ ID NO: 14, SEQ ID NO: 15, or SEQ ID NO: 16. It is expected that soluble ELP-2OST ELP-6OST, and ELP-3OST are all obtained in suitable quantities to both assemble into biomimetic condensates and synthesize heparan sulfate and/or heparin as a product.

Subsequently, the procedure of Example 6 or Example 13 is adapted to synthesize heparan sulfate and/or heparin. Condensation of ELP-2OST, ELP-6OST, and ELP-3OST is initiated upon adding all three enzymes to an aqueous composition and incubating the composition until mixed LLPS droplets are formed. Heparin synthesis is initiated by adding PAPS and completely desulfated, N-resulfated heparin, which can be chemically prepared from commercial samples of heparin. It is expected that the heparin product can be purified from the resulting composition, digested by one or more heparinase enzymes, and confirmed using strong anion exchange chromatography or mass spectrometry.

While several embodiments of the invention have been described, the invention can be further modified within the spirit and scope of this disclosure. Those skilled in the art will recognize, or be able to ascertain using no more than routine experimentation, numerous equivalents to the specific procedures, embodiments, claims, and examples described herein. As such, such equivalents are considered inside the scope of the invention, and this application is therefore intended to cover any variations, uses or adaptations of the invention using its general principles. Further, the invention is intended to cover such departures from the present disclosure as come within known or customary practice in the art to which this invention pertains and which fall within the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

The contents of all references, patents, and patent applications mentioned in this specification are hereby incorporated by reference, and shall not be construed as an admission that such reference is available as prior art to the present invention. All the incorporated publications and patent applications in this specification are indicative of the level of ordinary skill in the art to which this invention pertains, and are incorporated to the same extent as if each individual publication or patent application was specifically indicated and individually indicated by reference.

We claim:

1. A method for catalyzing the in vitro enzymatic synthesis of a biological product within a biomimetic condensate, the process comprising the following steps:
(a) providing an aqueous composition comprising one or more enzymes, the one or more enzymes having a biological activity, wherein the biological activity consists of producing a biological product upon reacting with one or more substrates;
(b) assembling the one or more enzymes into a biomimetic condensate having an internal portion and an external portion separated by a phase boundary,
wherein the one or more enzymes are contained within the internal portion, and wherein the internal portion is a liquid;
(c) introducing one or more substrates into the aqueous composition,
wherein the one or more substrates can freely traverse the phase boundary between the external portion and internal portion of the biomimetic condensate; and
(d) initiating a chemical reaction between at least one of the enzymes and at least one of the substrates to synthesize the biological product.

2. The method according to claim 1, wherein at least one of the enzymes is a fusion protein comprising the enzyme fused to a low-complexity phase separation domain selected from the group consisting of an intrinsically disordered region (IDR) and an elastin-like polypeptide (ELP) domain.

3. The method according to claim 2, wherein the phase separation domain is an IDR, wherein the amino acid sequence of the IDR is at least a fragment of a natural IDR present within the amino acid sequence an enzyme selected from the group consisting of fused in sarcoma (FUS) protein, TATA-box binding protein associated factor 15 (TAF), P-granule protein LAF-1 (LAF), Ddx4 helicase (DDX), and Tia1 cytotoxic granule-associated RNA binding protein (TIA).

4. The method according to claim 3, wherein the IDR comprises an amino acid sequence selected from the group consisting of SEQ ID NO: 18, SEQ ID NO: 19, SEQ ID NO: 20, SEQ ID NO: 21, SEQ ID NO: 22, SEQ ID NO: 23, SEQ ID NO: 24, and SEQ ID NO: 25.

5. The method according to claim 3, wherein the biological product is 2-(diethylamino)-acetic acid, the IDR is fused to an alcohol dehydrogenase (ALD) enzyme, and the one or more substrates comprise 2-(diethylamino)-ethanol and nicotinamide adenine dinucleotide (NAY).

6. The method according to claim 5, wherein the ALD comprises at least a fragment of an amino acid sequence selected from the group consisting of SEQ ID NO: 1, SEQ ID NO: 2, SEQ ID NO: 3, SEQ ID NO: 4, SEQ ID NO: 5, and SEQ ID NO: 6.

7. The method according to claim 5, wherein the biomimetic condensate further comprises Candida antarctica lipase B (CALB), the one or more substrates further comprises 2,6-xylidine, and the biological product is lidocaine.

8. The method according to claim 7, wherein the CALB comprises at least a fragment of the amino acid sequence, SEQ ID NO: 7.

9. The method according to claim 3; wherein:
the biological product is heparan sulfate,
the IDR is fused to one or more enzymes selected from the group consisting of hexuronyl 2-O sulfotransferase (2OST), glucosaminyl 6-O sulfotransferase (6OST), glucosaminyl 3-) sulfotransferase (3OST), and any combination thereof, and
the one or more substrates comprise PAPS and a sulfo group acceptor selected from the group consisting of N-sulfated heparan sulfate (N-HS), N-,2-O-sulfated heparan sulfate (N2-HS), N-,2-O,6-O-sulfated heparan sulfate (N26-HS), and any combination thereof.

10. The method according to claim 9, wherein:
the 2OST comprises at least a fragment of an amino acid sequence selected from the group consisting of SEQ ID NO: 8, SEQ ID NO: 9, and SEQ ID NO: 10;
the 6OST comprises at least a fragment of an amino acid sequence selected from the group consisting of SEQ ID NO: 11, SEQ 11) NO: 12, or SEQ ID NO: 13; and
the 3OST comprises at least a fragment of an amino acid sequence selected from the group consisting of SEQ ID NO: 14, SEQ ID NO: 15, or SEQ ID NO: 16.

11. The method according to claim 9, wherein 2OST is selected, and the one or more enzymes further comprises a glucuronyl C₅-epimerase (Epi) enzyme.

12. The method according to claim 11, wherein the heparin sulfate biological product is heparin.

13. The method according to claim 12, wherein each enzymatic reaction takes place within a different biomimetic condensate, wherein:
2OST and Epi are contained within a first biomimetic condensate;
3OST is contained within a second biomimetic condensate;
the first biomimetic condensate and the second biomimetic condensate abut each other;
6OST is contained either within the first biomimetic condensate or within a third biomimetic condensate that abuts the first biomimetic condensate and the second biomimetic condensate;
N-HS is introduced into the aqueous composition as a sulfo group acceptor for 2OST; and
the method further comprises the following steps:
within the first biomimetic condensate, synthesizing in situ a first intermediate product, N2-HS, as a sulfo group acceptor for 6OST;
synthesizing in situ a second intermediate product, N26-HS, as a sulfo group acceptor for 3OST; and
within the second biomimetic condensate, the synthesizing the heparin product.

14. A method for the in vitro enzymatic synthesis of a biological product within a biomimetic condensate, the process comprising the following steps:
(a) providing an aqueous composition comprising one or more scaffold proteins, the one or more scaffold protein comprising at least a fragment of a GKAP protein, at least a fragment of a Homer protein, and at least a fragment of a Shank protein,
wherein at least one of GKAP, Homer, Shank proteins is fused to an affinity tag;
(b) assembling the one or more scaffold proteins into a biomimetic condensate having an internal portion and an external portion separated by a phase boundary,
wherein the one or more scaffold proteins are contained within the internal portion, and
wherein the internal portion is a liquid;
(c) introducing one or more enzymes into the aqueous composition,
wherein at least one of the enzymes is fused to affinity tag partner having an equilibrium dissociation constant (K_{D}) with the affinity tag of less than 1 μM;
(d) binding the affinity tag partner with the affinity tag, thereby recruiting the one or more enzymes into the biomimetic condensate;
(e) introducing one or more substrates into the aqueous composition,
wherein the substrates can freely traverse the phase boundary between the external portion and internal portion of the biomimetic condensate; and
(f) initiating a chemical reaction between at least one of the enzymes and at least one of the substrates to synthesize the biological product.

15. The method according to claim 14, wherein:
the GKAP protein comprises at least a fragment of an amino acid sequence selected from the group consisting of SEQ ID NO: 27;
the Homer protein is a Homer3 isoform comprising at least a fragment of an amino acid sequence selected from the group consisting of SEQ ID NO: 29; and
the Shank protein is a Shank isoform comprising at least a fragment of an amino acid sequence selected from the group consisting of SEQ ID NO: 30.

16. The method according to claim 15, wherein:
the affinity tag is a RIAD peptide motif having the amino acid sequence of SEQ ID NO: 32; and
the affinity tag partner is a RIDD peptide motif having the amino acid sequence of SEQ ID NO: 33.

17. The method according to claim 16, wherein both Homer and Shank are fused with the RIAD peptide motif.

18. The method according to claim 17, wherein the biological product is 2-(diethylamino)-acetic acid, the affinity tag partner is fused to an ALD enzyme comprising at least a fragment of an amino acid sequence selected from the group consisting of SEQ ID NO: 1, SEQ ID NO: 2, SEQ ID NO: 3, SEQ ID) NO: 4, SEQ ID NO: 5, and SEQ ID NO: 6, and the one or more substrates comprise 2-(diethylamino)-ethanol and NAD.

19. The method according to claim 18, wherein the biomimetic condensate further comprises a CALB enzyme tagged with the affinity tag partner, wherein the CALB comprises at least a fragment of the amino acid sequence, SEQ ID NO: 7, the one or more substrates further comprises 2,6-xylidine, and the biological product is lidocaine.

20. The method according to claim 17, wherein the biological product is heparan sulfate, the affinity tag partner is fused to one or more enzymes selected from the group consisting of 2OST, 6OST, 3OST, and any combination thereof, and the one or more substrates comprise PAPS and a sulfo group acceptor selected from the group consisting of N-sulfated heparan sulfate (N-HS), N-,2-O-sulfated heparan sulfate (N2-HS), N-,2-O,6-O-sulfated heparan sulfate (N26-HS), and any combination thereof.
